(19) United States
(12) Patent Application Publication
(10) Pub. No.: US 2019/0091281 A1
(43) Pub. Date: Mar. 28, 2019

(54) DELIVERY PHARMACEUTICAL COMPOSITIONS INCLUDING PERMEATION ENHANCERS

(71) Applicant: AQUESTIVE THERAPEUTICS, INC., WARREN, NJ (US)

(72) Inventors: Stephen Paul Wargacki, Annandale, NJ (US); Rajesh Kumar Kainthan, Tappan, NY (US); Alexander Mark Schobel, Whitehouse Station, NJ (US)

(73) Assignee: AQUESTIVE THERAPEUTICS, INC., WARREN, NJ (US)

(21) Appl. No.: 16/143,036

(22) Filed: Sep. 26, 2018

Related U.S. Application Data
(60) Provisional application No. 62/563,534, filed on Sep. 26, 2017.

(51) Int. Cl. A61K 38/08; A61K 47/18; A61K 47/40; A61K 47/12; A61K 9/70

(52) U.S. Cl. CPC A61K 38/08; A61K 47/186; A61K 9/7007; A61K 47/12; A61K 47/40

(57) ABSTRACT

Pharmaceutical compositions having enhanced active component permeation properties are described.

### CLAIM OF PRIORITY

This application claims priority to U.S. Provisional Patent Application No. 62/563,534, filed Sep. 26, 2017, which is incorporated by reference in its entirety.

### TECHNICAL FIELD

This invention relates to pharmaceutical compositions.

### TECHNICAL FIELD

This invention relates to pharmaceutical compositions.

### BACKGROUND

Active ingredients, such as drugs or pharmaceuticals, are delivered to patients in deliberate fashion. Delivery of drugs or pharmaceuticals using film transdermally or transmucosally can require that the drug or pharmaceutical permeate or otherwise cross a biological membrane in an effective and efficient manner.

### SUMMARY

In general, a pharmaceutical composition includes a polymeric matrix, a pharmaceutically active component including a peptide in the polymeric matrix and a permeation enhancer including a surfactant.

In other embodiments, the pharmaceutically active component can be octreotide.

In certain embodiments, the surfactant is a cationic surfactant, the structure of which is

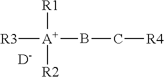

wherein:

A is either nitrogen or phosphorus;

C is a cleavable linkage;

B is a group connecting A with C and can be an alkylene, alkenylene, cycloalkylene or aralkylene group and its derivatives optionally containing one or more hetero atoms;

each R¹, R² and R³, independently, is selected from the group consisting of hydrogen, alkyl, alkenyl, alkynyl, cycloalkyl and aralkyl group optionally having one or more heteroatoms;

R⁴ is selected from the group consisting of alkyl, alkenyl, alkynyl, cycloalkyl and aralkyl group optionally having one or more heteroatoms;

D− is an anionic counter ion to A⁺.

In certain embodiments, each of R¹, R² and R³, independently, can be each independently a C₁₋₁₀ alkyl, C₂₋₁₀ alkenyl, C₂₋₁₀ alkynyl, C₃₋₁₀ cycloalkyl, C₄₋₁₀ aralkyl group or derivatives thereof optionally having one or more heteroatoms.

In certain embodiments, B can be a C₁₋₂₀ alkylene, C₂₋₂₀ alkenylene, C₂₋₂₀ alkynylene, C₃₋₂₀ cycloalkylene, C₄₋₂₀ aralkylene group or derivative thereof optionally having one or more heteroatoms.

In certain embodiments, R⁴ can be a C₁₋₃₀ alkyl, C₂₋₃₀ alkenyl, C₂₋₃₀ alkynyl, C₃₋₃₀ cycloalkyl, C₄₋₃₀ aralkyl group or their derivatives optionally having one or more heteroatoms.

In certain embodiments, C can be a degradable group through acid/base hydrolysis, enzymatic reaction or radical cleavage. For example, C can be selected from the group, but not limited to, consisting of a carbonate linkage, an amide linkage, an ester linkage, acetal linkage, hemiacetal linkage, orthoester linkage, carbamide, sulphonate, phosphonate, thioester, urea, isocyanate linkages, hydrozone, disulfidelinkages and combinations thereof.

In certain embodiments, D− can be chloride, bromide, iodide, sulfate, sulfonate, carbonate, or hydroxide ion.

In certain embodiments, the surfactant can include a plurality of amino groups, for example, 2, 3, 4 or more amino groups as substituents.

In certain embodiments, surfactant can include dodecyltrimethylammonium bromide.

The cationic surfactant can include hexadecyltrimethylammonium bromide (HDTMAB or CTAB).

The cationic surfactant can include benzalkonium chloride (BAC).

In certain embodiments, a permeation enhancer such as a cationic surfactant can be combined with a non-ionic or anionic surfactant.

In other embodiments, a cationic surfactant can be combined with a chelator. In yet other embodiments, the surfactant can be combined with a cyclodextrin.

In other embodiments, the surfactant can be combined with a fatty acid.

In certain embodiments, the permeation enhancer can be biodegradable.

In other embodiments, the permeation enhancer can be glycine betaine derivative

In some examples, octreotide is delivered from a pharmaceutical composition film.

For example, the octreotide can be delivered from a pharmaceutical film having an occlusive layer and an active layer. The octreotide and permeation enhancer can be embedded in an active layer of a pharmaceutical composition film.

In certain embodiments, the permeation activity of (dodecyltrimethylammonium bromide) DDTMAB is concentration dependent as shown in an ex vivo permeation model. For example, the permeation enhancer can be 5% wt DDTMAB. The permeation enhancer can also be 1% wt DDTMAB, 0.5% wt DDTMAB, or 0.1% wt DDTMAB.

In certain embodiments, a permeation enhancer can be 10% wt glycine betaine ester (C12). The permeation enhancer can also be 5% wt glycine betaine, 0.5% wt glycine betaine, or 0.1 5% wt glycine betaine esters.

In certain embodiments, the polymer matrix can include a polyethylene oxide.

In certain embodiments, the polymer matrix can include a cellulosic polymer is selected from the group of: hydroxypropylmethyl cellulose, hydroxyethyl cellulose, hydroxyethylmethyl cellulose, hydroxypropyl cellulose, and carboxymethyl cellulose and sodium carboxymethylcellulose.

In certain embodiments, the polymeric matrix can include hydroxypropyl methylcellulose.

In certain embodiments, the polymeric matrix can include polyethylene oxide and hydroxypropyl methylcellulose.

In certain embodiments, the polymeric matrix can include polyethylene oxide and polyvinyl pyrrolidone.

In certain embodiments, the polymeric matrix can include polyethylene oxide and a poly-saccharide.

In certain embodiments, the polymeric matrix can include polyethylene oxide, hydroxypropyl methylcellulose and a polysaccharide.

In certain embodiments, the polymeric matrix can include polyethylene oxide, hydroxypropyl methylcellulose, polysaccharide and polyvinylpyrrolidone.

In certain embodiments, the polymeric matrix can include at least one polymer selected from the group of: pullulan, polyvinyl pyrrolidone, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, ethylene oxide-propylene oxide co-polymers, collagen, albumin, poly-amino acids, polyphosphazenes, polysaccharides, chitin, chitosan, and derivatives thereof.

The polymeric matrix can include a dendritic polymer. The polymeric matrix can include a hyperbranched polymer.

A method of making a pharmaceutical composition can include mixing a permeation enhancer including a surfactant with a pharmaceutically active component including octreotide and embedding the pharmaceutically active component including octreotide in a pharmaceutical film.

In general, a pharmaceutical composition can be dispensed from a device. The device can include a housing that holds an amount of a pharmaceutical composition, including a polymeric matrix, a pharmaceutically active component including octreotide in the polymeric matrix, and a permeation enhancer including a surfactant, and an opening that dispenses a predetermined amount of the pharmaceutical composition.

In certain embodiments, the pharmaceutical composition can include a stabilizer.

In yet another aspect, the pharmaceutical composition has a suitable nontoxic, nonionic alkyl glycoside having a hydrophobic alkyl group joined by a linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) a nitric oxide donor compound; (vi) a long chain amphipathic molecule; (vii) a small hydrophobic penetration enhancer, (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced mucosal delivery, wherein the formulation of the compound with the transmucosal delivery-enhancing agents provides for increased bioavailability of the compound in a blood plasma of a subject.

In certain embodiments, a pharmaceutical composition can include a polymeric matrix; a pharmaceutically active component in the polymeric matrix; and an interacter that creates increased blood flow or enables a flushing of the tissue to modify transmucosal uptake of the pharmaceutically active component.

In certain embodiments, a pharmaceutical composition can include a polymeric matrix; a pharmaceutically active component in the polymeric matrix; and an interacter that has a positive or negative heat of solution which are used as aids to modify (increase or decrease) transmucosal uptake.

In other embodiments, a pharmaceutical composition includes a polymeric matrix, a pharmaceutically active component in the polymeric matrix, and an interacter, the composition contained in a multilayer film having at least one side where the edges are coterminous.

In general, a method of treating a medical condition can include administering a pharmaceutical composition including a polymeric matrix, an effective amount of a pharmaceutically active component including octreotide in the polymeric matrix, and a permeation enhancer including a surfactant. Octreotide, can be used to inhibits the release of growth hormone from the pituitary gland. It can be used for treatment of growth hormone producing tumors (e.g., acromegaly and gigantism), pituitary tumors that secrete thyroid stimulating hormone (e.g., thyrotropinoma), diarrhea and flushing episodes associated with carcinoid syndrome, or diarrhea in people with vasoactive intestinal peptide-secreting tumors (VIPomas). It can also be used as treatment for management of acute hemorrhage from esophageal varices in liver cirrhosis. Other aspects, embodiments, and features will be apparent from the following description, the drawings, and the claims.

### BRIEF DESCRIPTION OF THE FIGURES

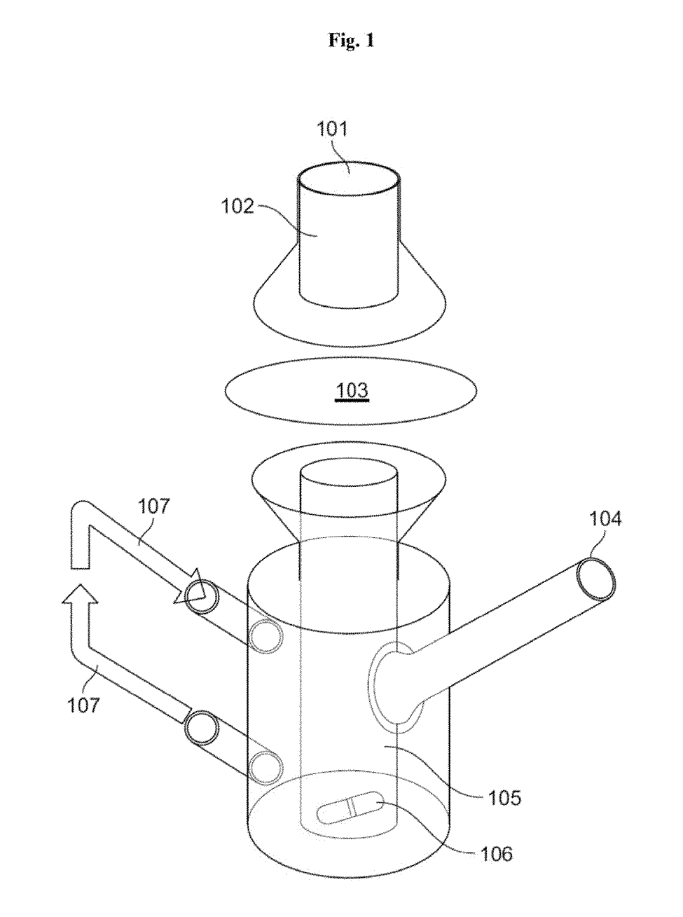

Referring to FIG. 1, a Franz diffusion cell 100 includes a donor compound 101, a donor chamber 102, a membrane 103, sampling port 104, receptor chamber 105, stir bar 106, and a heater/circulator 107.

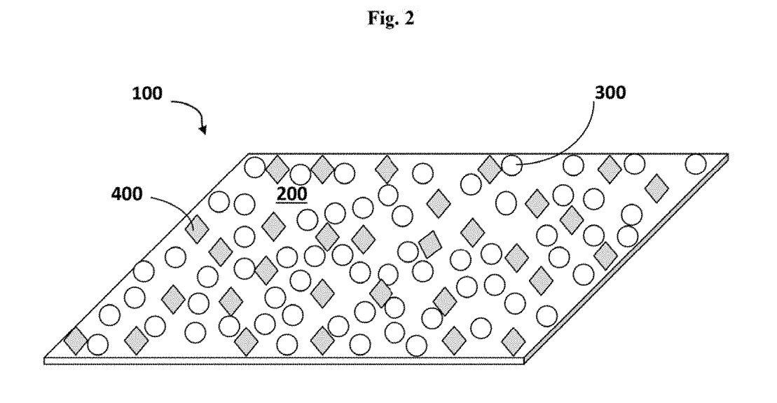

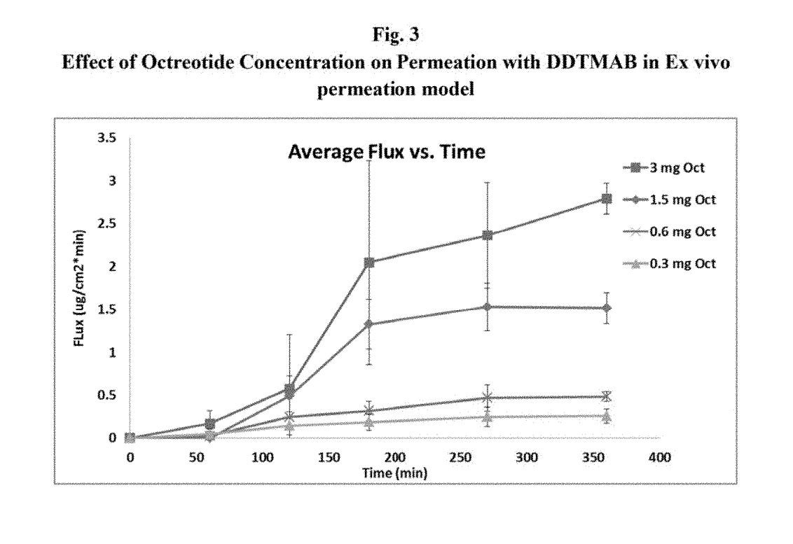

Referring to FIG. 2, a pharmaceutical composition is a film 100 comprising a polymeric matrix 200, the pharmaceutically active component 300 being dispersed in the polymeric matrix. The film can include a permeation enhancer 400 which can be a surfactant.

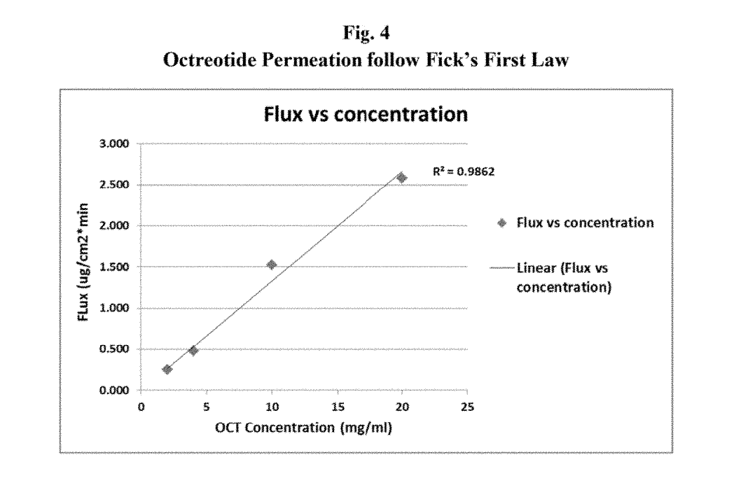

Referring to FIG. 3, this graph shows the effect of octreotide concentration on permeation with DDTMAB.

Referring to FIG. 4, this graph shows octreotide permeation according to Fick's first law of diffusion.

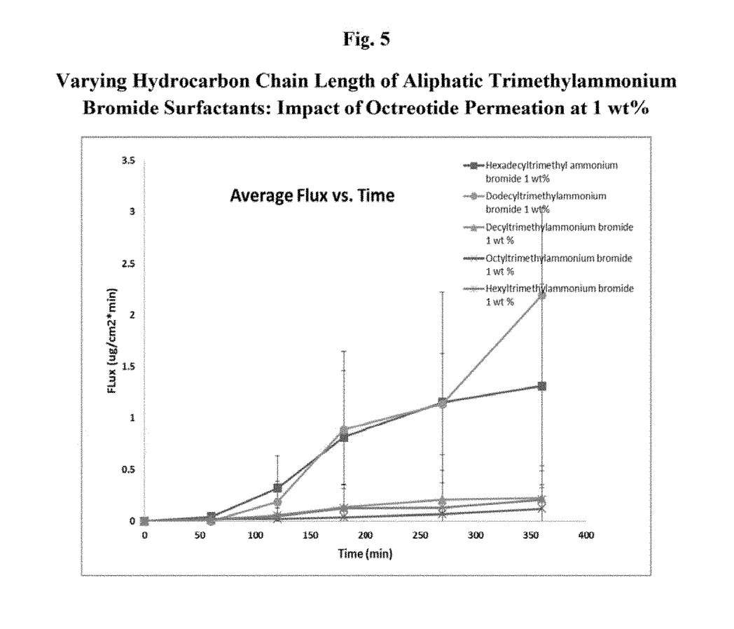

Referring to FIG. 5, this graph shows the structure-activity relationship of aliphatic trimethyl-ammonium bromide surfactants.

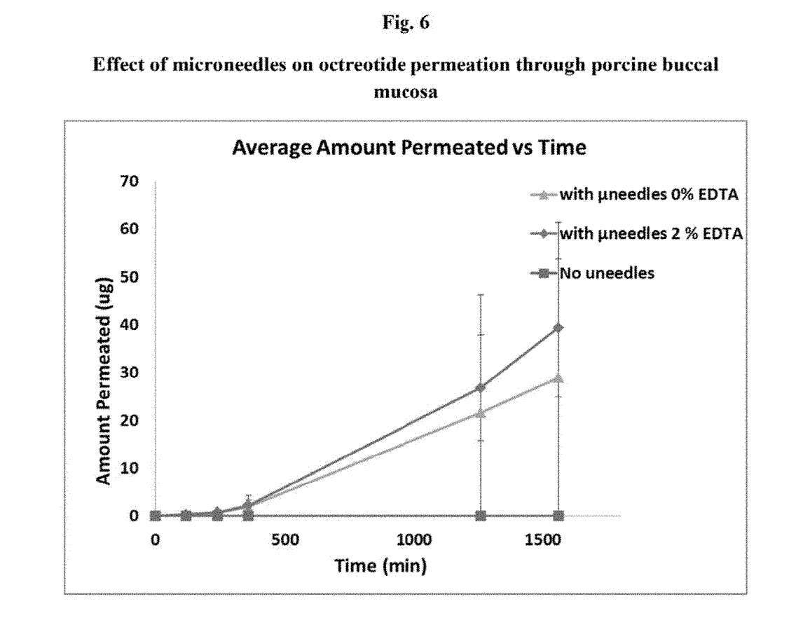

Referring to FIG. 6, this graph shows the microneedle impact on octreotide permeation with porcine buccal tissue.

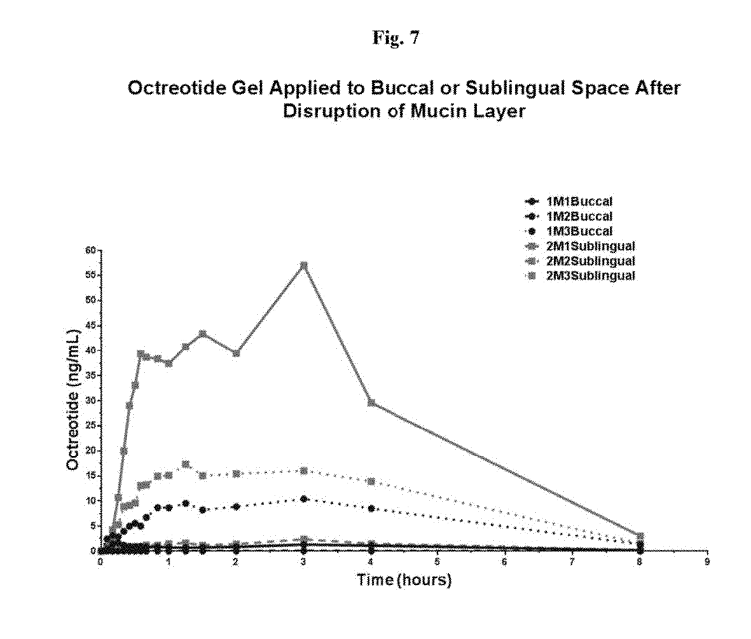

Referring to FIG. 7, this graph shows the results from a preclinical study in which octreotide solution was applied to a buccal and sublingual space after microneedle application

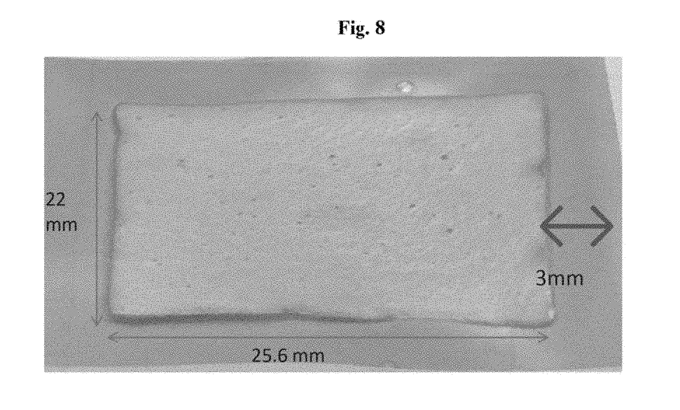

Referring to FIG. 8, this image shows a pharmaceutical composition bilayer film with octreotide as the active pharmaceutical ingredient.

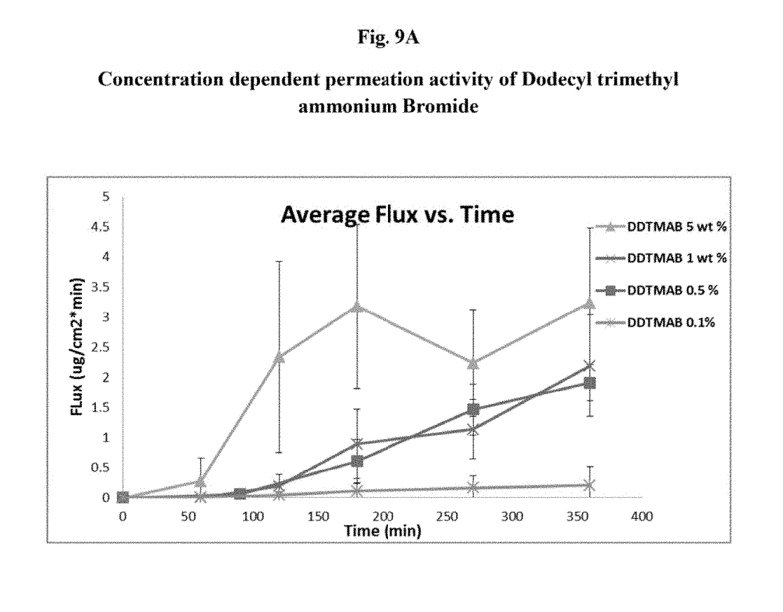

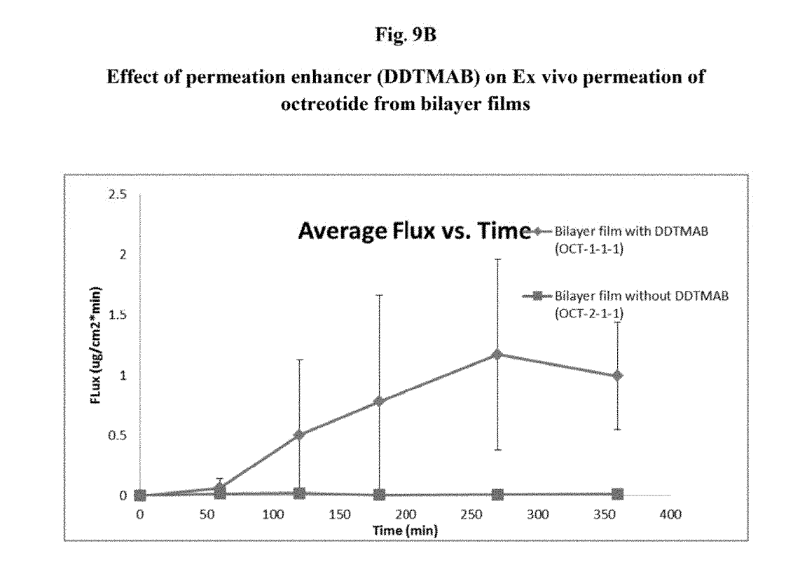

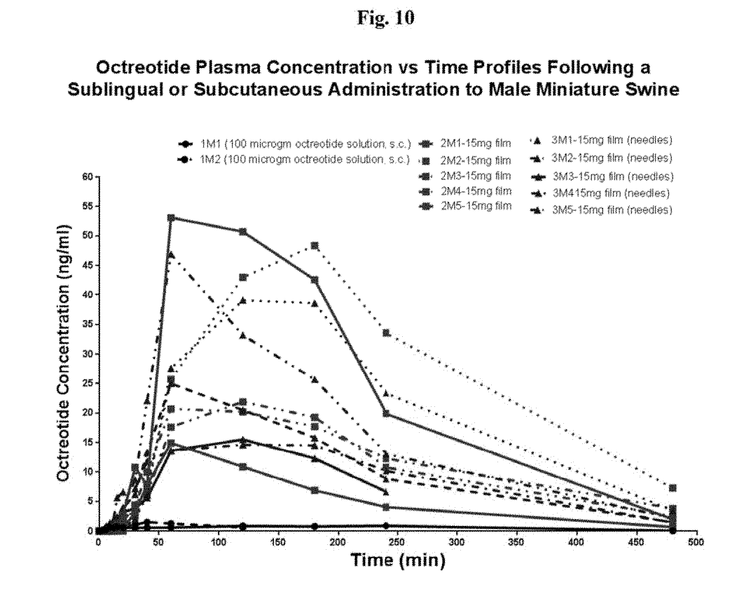

Referring to FIG. 9A, this graph indicates Concentration dependent permeation activity of Dodecyl trimethyl ammonium Bromide. Referring to FIG. 9B, this graph indicates the Effect of permeation enhancer (DDTMAB) on Ex vivo permeation of octreotide from bilayer films Referring to FIG. 10, this graph indicates octreotide plasma concentration following sublingual or subcutaneous administration.

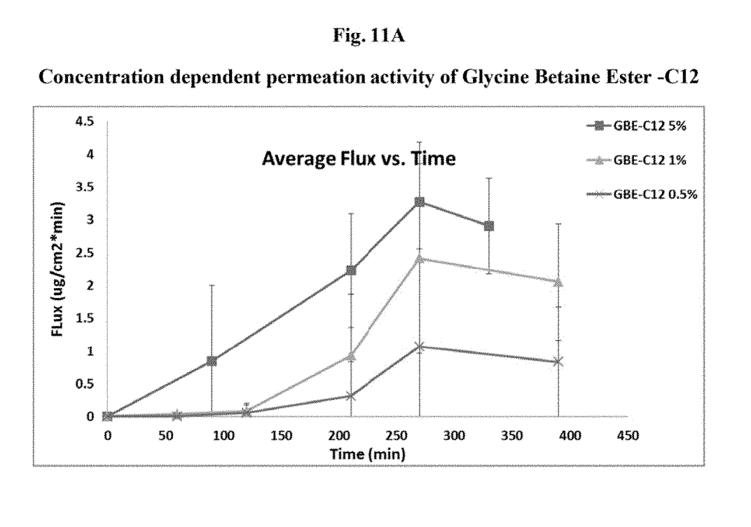

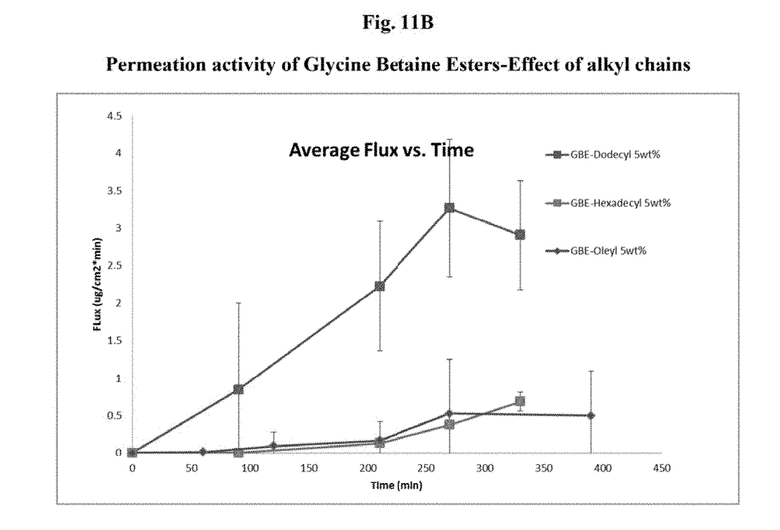

Referring to FIG. 11A, the graph shows concentration dependent permeation activity of glycine betaine ester.

Referring to FIG. 11B, the graph shows the effect of alkyl chains on the permeation activity of glycine betaine esters.

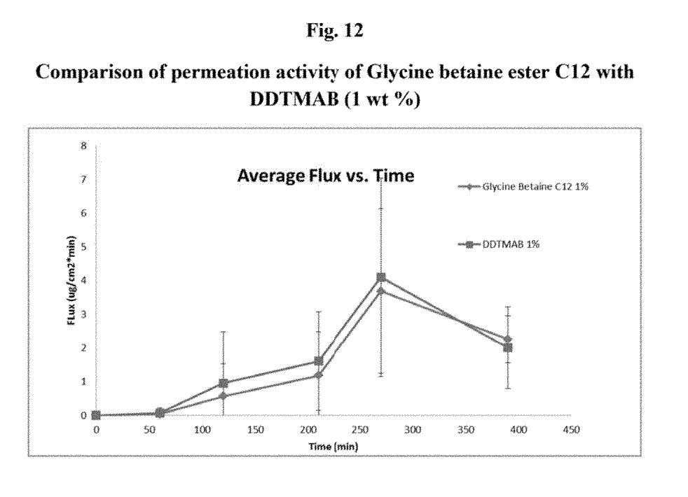

Referring to FIG. 12, the graph shows a comparison of permeation activity of glycine betaine ester C12 with DDTMAB.

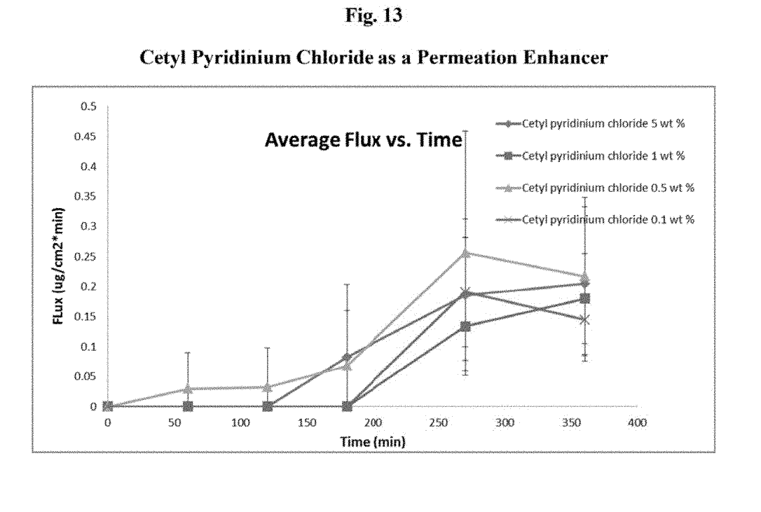

Referring to FIG. 13, the graph shows the effect of cetyl pyridium chloride Tetrahexyl Ammonium Bromide on Octreotide Permeation in Ex vivo Permeation Model.

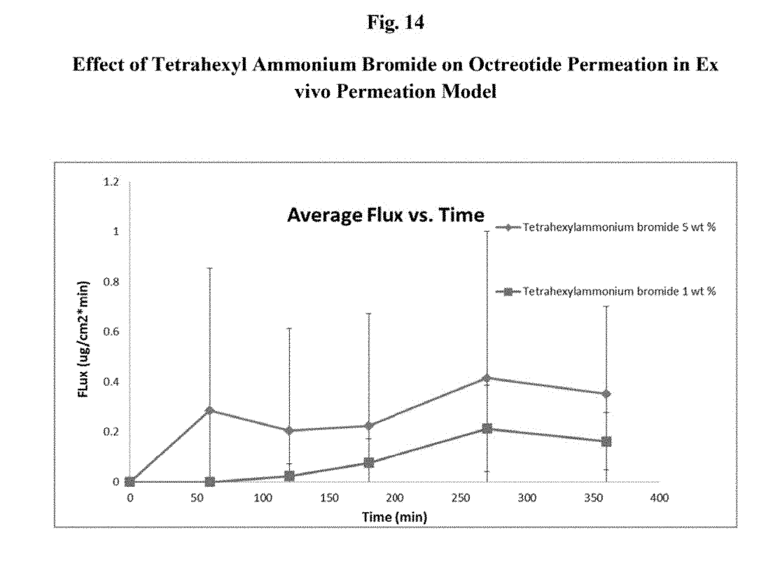

Referring to FIG. 14, the graph shows the effect of Tetrahexyl Ammonium Bromide on Octreotide Permeation in Ex vivo Permeation Model.

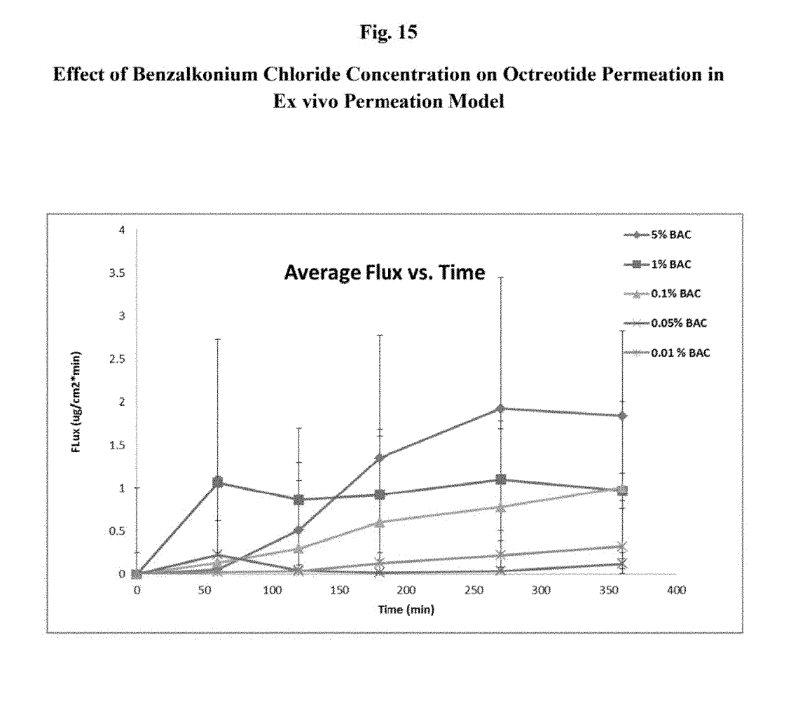

Referring to FIG. 15, the graph shows Effect of Benzalkonium Chloride Concentration on Octreotide Permeation in Ex vivo Permeation Model benzalkonium chloride as a permeation enhancer.

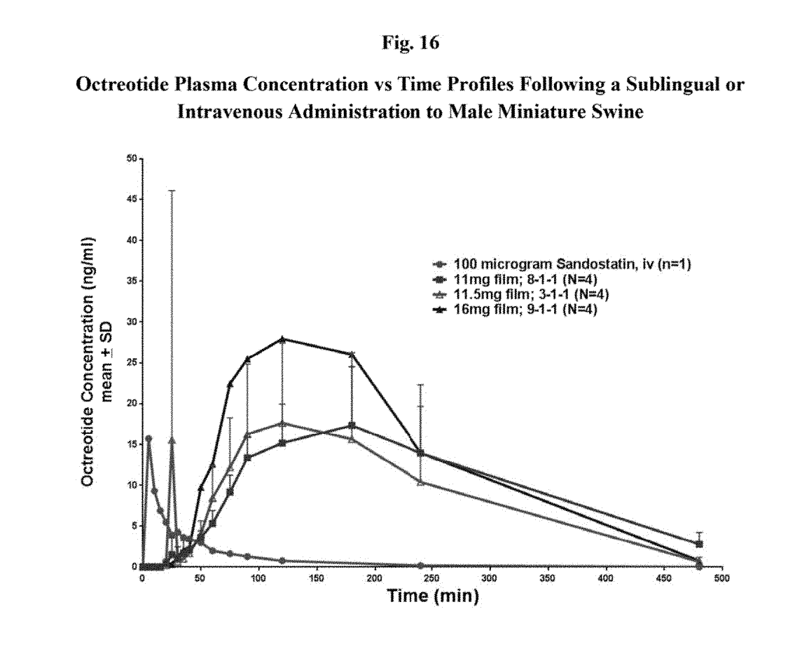

Referring to FIG. 16, the graph shows octreotide plasma concentration (ng/ml) vs. time profiles following sublingual or intravenous (IV) administration to male miniature swine.

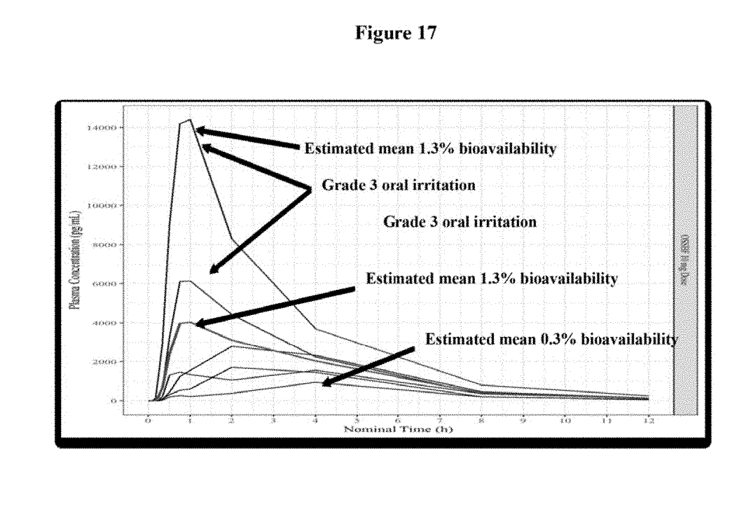

Referring to FIG. 17, the graph shows arm #1 of the human study with 10 mg octreotide/25 mg BAC.

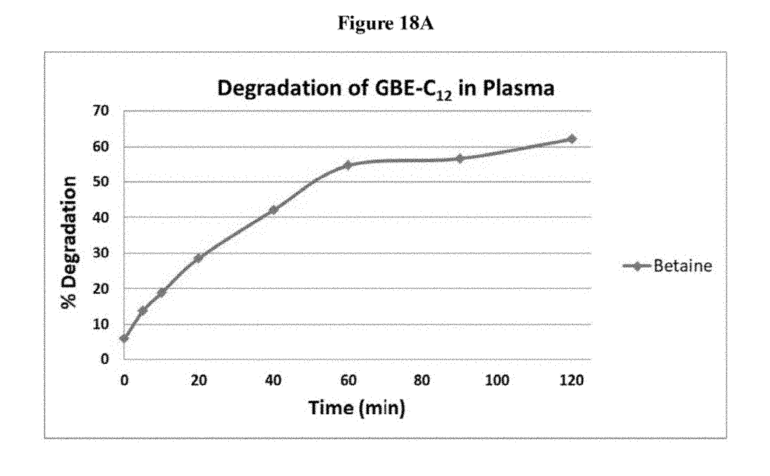

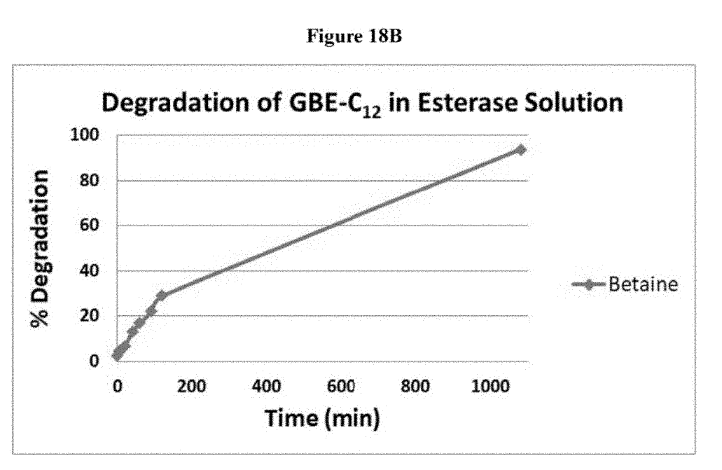

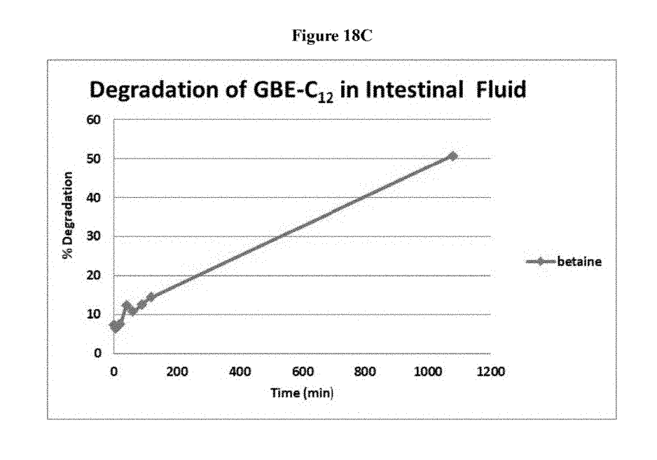

Referring to FIGS. 18A-18C, the graphs show the results of degradation studies of GBE-C12 in gastric fluid, intestinal fluid, and tissue extract, respectively.

### DETAILED DESCRIPTION

Mucosal surfaces, such as the oral mucosa, are a convenient route for delivering drugs to the body due to the fact that they are highly vascularized and permeable, providing increased bioavailability and rapid onset of action because it does not pass through the digestive system and thereby avoids first pass metabolism. In particular, the buccal and sublingual tissues offer advantageous sites for drug delivery because they are highly permeable regions of the oral mucosa, allowing drugs diffusing from the oral mucosa to have direct access to systemic circulation. This also offers increased convenience and therefore increased compliance in patients. For certain drugs, or pharmaceutically active components, a permeation enhancer can help to overcome the mucosal barrier and improve permeability. Permeation enhancers reversibly modulate the penetrability of the barrier layer in favor of drug absorption. Permeation enhancers facilitate transport of molecules through the epithelium. Absorption profiles and their rates can be controlled and modulated by a variety of parameters, such as but not limited to film size, drug loading, enhancer type/loading, polymer matrix release rate and mucosal residence time.

A pharmaceutical composition can be designed to deliver a pharmaceutically active component in a deliberate and tailored way. However, solubility and permeability of the pharmaceutically active component in vivo, in particular, in the mouth of a subject, can vary tremendously. A particular class of permeation enhancer can improve the uptake and bioavailability of the pharmaceutically active component in vivo. In particular, when delivered to the mouth via a film, the permeation enhancer can improve the permeability of the pharmaceutically active component through the mucosa and into the blood stream of the subject. The permeation enhancer can improve absorption rate and amount of the pharmaceutically active component by 10%, 20%, 30%, 40%, 50%, 60%, 70%, 80%, 90%, 100%, 150%, 200% or more depending on the other components in the composition.

In certain embodiments, a pharmaceutical composition has a suitable nontoxic, nonionic alkyl glycoside having a hydrophobic alkyl group joined by a linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a small hydrophobic penetration enhancer, (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced mucosal delivery, wherein the formulation of the compound with the transmucosal delivery-enhancing agents provides for increased bioavailability of the compound in a blood plasma of a subject.

“Alkyl” means a straight chain or branched, noncyclic or cyclic, saturated aliphatic hydrocarbon containing from 1 to 24 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. It has been found that charged lipids comprising unsaturated alkyl chains are particularly useful for forming lipid nucleic acid particles with increased membrane fluidity. See, e.g., U.S. Pat. App. Pub. 2013/0338210, which is incorporated by reference herein.

### Permeation Enhancers

Penetration enhancers have been described in J. Nicolazzo, et al., J. of Controlled Disease, 105 (2005) 1-15, which is incorporated by reference herein. There are many reasons why the oral mucosa might be an attractive site for the delivery of therapeutic agents into the systemic circulation. Due to the direct drainage of blood from the buccal epithelium into the internal jugular vein first-pass metabolism in the liver and intestine may be avoided. The first-pass effect can be a major reason for the poor bioavailability of some compounds when administered orally. Additionally, the mucosa lining the oral cavity is easily accessible, which ensures that a dosage form can be applied to the required site and can be removed easily in the case of an emergency. However, like the skin, the buccal mucosa acts as a barrier to the absorption of xenobiotics, which can hinder the permeation of compounds across this tissue. Consequently, the identification of safe and effective penetration enhancers has become a major goal in the quest to improve oral mucosal drug delivery.

Chemical penetration enhancers are substances that control the permeation rate of a coadministered drug through a biological membrane. While extensive research has focused on obtaining an improved understanding of how penetration enhancers might alter intestinal and transdermal permeability, far less is known about the mechanisms involved in buccal penetration enhancement.

The buccal mucosa delineates the inside lining of the cheek as well as the area between the gums and upper and lower lips and it has an average surface area of 100 cm². The surface of the buccal mucosa consists of a stratified squamous epithelium which is separated from the underlying connective tissue (lamina propria and submucosa) by an undulating basement membrane (a continuous layer of extracellular material approximately 1-2 Am in thickness). This stratified squamous epithelium consists of differentiating layers of cells which change in size, shape, and content as they travel from the basal region to the superficial region, where the cells are shed. There are approximately 40-50 cell layers, resulting in a buccal mucosa which is 500-600 Am thick.

The permeability of the buccal mucosa is greater than that of the skin, but less than that of the intestine. The differences in permeability are the result of structural differences between each of the tissues. The absence of organized lipid lamellae in the intercellular spaces of the buccal mucosa results in a greater permeability of exogenous compounds, compared to keratinized epithelia of the skin; while the increased thickness and lack of tight junctions results in the buccal mucosa being less permeable than intestinal tissue.

The primary barrier properties of the buccal mucosa have been attributed to the upper one-third to one-quarter of the buccal epithelium. Researchers have learned that beyond the surface epithelium, the permeability barrier of nonkeratinized oral mucosa could be attributed to contents extruded from the membrane-coating granules into the epithelial intercellular spaces.

The intercellular lipids of the nonkeratinized regions of the oral cavity are of a more polar nature than the lipids of the epidermis, palate, and gingiva, and this difference in the chemical nature of the lipids may contribute to the differences in permeability observed between these tissues. Consequently, it appears that it is not only the greater degree of intercellular lipid packing in the stratum corneum of keratinized epithelia that creates a more effective barrier, but also the chemical nature of the lipids present within that barrier.

The existence of hydrophilic and lipophilic regions in the oral mucosa has led researchers to postulate the existence of two routes of drug transport through the buccal mucosa-paracellular (between the cells) and transcellular (across the cells).

Since drug delivery through the buccal mucosa is limited by the barrier nature of the epithelium and the area available for absorption, various enhancement strategies are required in order to deliver therapeutically relevant amounts of drug to the systemic circulation. Various methods, including the use of chemical penetration enhancers, prodrugs, and physical methods may be employed to overcome the barrier properties of the buccal mucosa.

A chemical penetration enhancer, or absorption promoter, is a substance added to a pharmaceutical formulation in order to increase the membrane permeation or absorption rate of the coadministered drug. This can be donewithout damaging the membrane and/or causing toxicity. There have been many studies investigating the effect of chemical penetration enhancers on the delivery of compounds across the skin, nasal mucosa, and intestine. In recent years, more attention has been given to the effect of these agents on the permeability of the buccal mucosa. Since permeability across the buccal mucosa is considered to be a passive diffusion process the steady state flux (Jss) should increase with increasing donor chamber concentration (CD) according to Fick's first law of diffusion.

Surfactants and bile salts have been shown to enhance the permeability of various compounds across the buccal mucosa, both in vitro and in vivo. The data obtained from these studies strongly suggest that the enhancement in permeability is due to an effect of the surfactants on the mucosal intercellular lipids. Surfactants typically function by perturbation of intercellular lipids and protein domains. Surfactants can be cationic, nonionic or anionic. Examples of cationic surfactants include DDTMA, CTAB, and BAC. Examples of anionic surfactants include (Sodium glycodeoxycholate (GDC) and (Sodium Deoxycholate (DOC). Examples of nonionic surfactants include Polaxamer F127, Azone/Dimethyl cyclodextrin (DMCD), Peceol, Labrasol, and TDM.

Fatty acids have been shown to enhance the permeation of a number of drugs through the skin, and this has been shown by DSC and FTIR to be related to an increase in the fluidity of intercellular lipids. An example of a fatty acid is oleic acid.

Cyclodextrins have also been used to enhance permeation by the inclusion of complexes and extraction of membrane compounds. Examples of cyclodextrins include dimethyl-cyclodextrin and beta-cyclodextrin.

Chelators have also been used to enhance permeation by interfering with Ca2+ calcium ions. Examples of chelators include EDTA and EGTA.

Additionally, pretreatment with ethanol has been shown to enhance the permeability of tritiated water and albumin across ventral tongue mucosa, and to enhance caffeine permeability across porcine buccal mucosa. There are also several reports of the enhancing effect of Azonel on the permeability of compounds through oral mucosa. Further, chitosan, a biocompatible and biodegradable polymer, has been shown to enhance drug delivery through various tissues, including the intestine and nasal mucosa.

Oral transmucosal drug delivery (OTDD) is the administration of pharmaceutically active agents through the oral mucosa to achieve systemic effects. Permeation pathways and predictive models for OTDD are described, e.g. in M. Sattar, Oral transmucosal drug delivery—Current status and future prospects, Int'l. Journal of Pharmaceutics, 47(2014) 498-506, which is incorporated by reference herein. OTDD continues to attract the attention of academic and industrial scientists. Despite limited characterization of the permeation pathways in the oral cavity compared with skin and nasal routes of delivery, recent advances in our understanding of the extent to which ionized molecules permeate the buccal epithelium as well as the emergence of new analytical techniques to study the oral cavity, and the progressing development of in silico models predictive of buccal and sublingual permeation are encouraging.

In order to deliver broader classes of drugs across the buccal mucosa, reversible methods of reducing the barrier potential of this tissue should be employed. This requisite has fostered the study of penetration enhancers that will safely alter the permeability restrictions of the buccal mucosa. It has been shown that buccal penetration can be improved by using various classes of transmucosal and transdermal penetration enhancers such as bile salts, surfactants, fatty acids and their derivatives, chelators, cyclodextrins and chitosan. Chemicals used for the drug permeation enhancement can include bile salts.

In vitro studies on enhancing effect of bile salts on the buccal permeation of compounds is discussed in Sevda Senel, Drug permeation enhancement via buccal route: possibilities and limitations, Journal of Controlled Release 72 (2001) 133-144, which is incorporated by reference herein. That article also discusses recent studies on the effects of buccal epithelial permeability of dihydroxy bile salts, sodium glycodeoxycholate (GDC) and sodium taurodeoxycholate (TDC) and tri-hydroxy bile salts, sodium glycocholate (GC) and sodium taurocholate (TC) at 100 mM concentration including permeability changes correlated with the histological effects. Fluorescein isothiocyanate (FITC), morphine sulfate were each used as the model compound.

Chitosan has also been shown to promote absorption of small polar molecules and peptide/protein drugs through nasal mucosa in animal models and human volunteers. Other studies have shown an enhancing effect on penetration of compounds across the intestinal mucosa and cultured Caco-2 cells.

The permeation enhancer can be a phytoextract. A phytoextract can be an essential oil or composition including essential oils extracted by distillation of the plant material. In certain circumstances, the phytoextract can include a synthetic analogues of the compounds extracted from the plant material (i.e., compounds made by organic synthesis). The phytoextract can include a phenylpropanoid, for example, phenyl alanine, eugenol, eugenol acetate, a cinnamic acid, a cinnamic acid ester, a cinnamic aldehyde, a hydrocinnamic acid, chavicol, or safrole, or a combination thereof. The phytoextract can be an essential oil extract of a clove plant, for example, from the leaf, stem or flower bud of a clove plant. The clove plant can be Syzygium aromaticum. The phytoextract can include 60-95% eugenol, for example, 80-95% eugenol. The extract can also include 5% to 15% eugenol acetate. The extract can also include caryophyllene. The extract can also include up to 2.1% α-humulen. Other volatile compounds included in lower concentrations in clove essential oil can be β-pinene, limonene, farnesol, benzaldehyde, 2-heptanone and ethyl hexanoate.

Other permeation enhancers may be added to improve absorption of the drug. Suitable permeation enhancers include natural or synthetic bile salts such as sodium fusidate; glycocholate or deoxycholate; fatty acids and derivatives such as sodium laurate, oleic acid, oleyl alcohol, monoolein, and palmitoylcarnitine; chelators such as disodium EDTA, EGTA, sodium citrate and sodium laurylsulfate, azone, sodium cholate, sodium 5-methoxysalicylate, sorbitan laurate, glyceryl monolaurate, octoxynonyl-9, laureth-9, polysorbates, sterols, or glycerides, such as caprylocaproyl polyoxylglycerides, e.g., Labrasol.

Some natural products of plant origin have been known to have a vasodilatory effect. There are several mechanisms or modes by which plant-based products can evoke vasodilation. For review, see McNeill, J. R. and Jurgens, T. M., Can. J. Physiol. Pharmacol. 84:803-821 (2006), which is incorporated by reference herein. Specifically, vasorelaxant effects of eugenol have been reported in a number of animal studies. See, Lahlou, S., et al., J. Cardiovasc. Pharmacol. 43:250-57 (2004), Damiani, C. E. N., et al., Vascular Pharmacol. 40:59-66 (2003), Nishijima, H., et al., Japanese J. Pharmacol. 79:327-334 (1998), and Hume W. R., J. Dent Res. 62(9):1013-15 (1983), each of which is incorporated by reference herein. Calcium channel blockade was suggested to be responsible for vascular relaxation induced by a plant essential oil, or its main constituent, eugenol. See, Interaminense L. R. L. et al., Fundamental & Clin. Pharmacol. 21: 497-506 (2007), which is incorporated by reference herein.

Fatty acids can be used as inactive ingredients in drug preparations or drug vehicles. Fatty acids can also be used as formulation ingredients due to their certain functional effects and their biocompatible nature. Fatty acid, both free and as part of complex lipids, are major metabolic fuel (storage and transport energy), essential components of all membranes and gene regulators. For review, see Rustan A. C. and Drevon, C. A., Fatty Acids: Structures and Properties, Encyclopedia of Life Sciences (2005), which is incorporated by reference herein. There are two families of essential fatty acids that are metabolized in the human body: ω-3 and ω-6 polyunsaturated fatty acids (PUFAs). If the first double bond is found between the third and the fourth carbon atom from the ω carbon, they are called ω-3 fatty acids. If the first double bond is between the sixth and seventh carbon atom, they are called ω-6 fatty acids. PUFAs are further metabolized in the body by the addition of carbon atoms and by desaturation (extraction of hydrogen). Linoleic acid, which is a ω-6 fatty acid, is metabolized to γ-linolenic acid, dihomo-γ-linolinic acid, arachidonic acid, adrenic acid, tetracosatetraenoic acid, tetracosapentaenoic acid and docosapentaenoic acid. α-linolenic acid, which is a ω-3 fatty acid is metabolized to octadecatetraenoic acid, eicosatetraenoic acid, eicosapentaenoic acid (EPA), docosapentaenoic acid, tetracosapentaenoic acid, tetracosahexaenoic acid and docosahexaenoic acid (DHA).

It has been reported that fatty acids, such as palmitic acid, oleic acid, linoleic acid and eicosapentaenoic acid, induced relaxation and hyperpolarization of porcine coronary artery smooth muscle cells via a mechanism involving activation of the Na⁺K⁺-APTase pump and the fatty acids with increasing degrees of cis-unsaturation had higher potencies. See, Pomposiello, S. I. et al., Hypertension 31:615-20 (1998), which is incorporated by reference in its herein. Interestingly, the pulmonary vascular response to arachidonic acid, a metabolite of linoleic acid, can be either vasoconstrictive or vasodilative, depending on the dose, animal species, the mode of arachidonic acid administration, and the tones of the pulmonary circulation. For example, arachidonic acid has been reported to cause cyclooxygenase-dependent and -independent pulmonary vasodilation. See, Feddersen, C. O. et al., J. Appl. Physiol. 68(5):1799-808 (1990); and see, Spannhake, E. W., et al., J. Appl. Physiol. 44:397-495 (1978) and Wicks, T. C. et al., Circ. Res. 38:167-71 (1976), each of which is incorporated by reference herein.

Many studies have reported effects of EPA and DHA on vascular reactivity after being administered as ingestible forms. Some studies found that EPA-DHA or EPA alone suppressed the vasoconstrictive effect of norepinephrine or increased vasodilatory responses to acetylcholine in the forearm microcirculation. See, Chin, J. P. F, et al., Hypertension 21:22-8 (1993), and Tagawa, H. et al., J Cardiovasc Pharmacol 33:633-40 (1999), each of which is incorporated by reference herein. Another study found that both EPA and DHA increased systemic arterial compliance and tended to reduce pulse pressure and total vascular resistance. See, Nestel, P. et al., Am J. Clin. Nutr. 76:326-30 (2002), which is incorporated by reference herein. Meanwhile, a study found that DHA, but not EPA, enhanced vasodilator mechanisms and attenuates constrictor responses in forearm microcirculation in hyperlipidemic overweight men. See, Mori, T. A., et al., Circulation 102:1264-69 (2000), which is incorporated by reference herein. Another study found vasodilator effects of DHA on the rhythmic contractions of isolated human coronary arteries in vitro. See Wu, K.-T. et al., Chinese J. Physiol. 50(4):164-70 (2007), which is incorporated by reference herein.

Adrenergic receptors (or adrenoceptors) are a class of G protein-coupled receptors that are a target of catecholamines, especially norepinephrine (noradrenaline) and epinephrine (adrenaline). Epinephrine (adrenaline) reacts with both α- and β-adrenoceptors, causing vasoconstriction and vasodilation, respectively. Although a receptors are less sensitive to epinephrine, when activated, they override the vasodilation mediated by β-adrenoceptors because there are more peripheral α1 receptors than β-adrenoceptors. The result is that high levels of circulating epinephrine cause vasoconstriction. At lower levels of circulating epinephrine, β-adrenoceptor stimulation dominates, producing vasodilation followed by decrease of peripheral vascular resistance. The α1-adrenoreceptor is known for smooth muscle contraction, mydriasis, vasoconstriction in the skin, mucosa and abdominal vicera and sphincter contraction of the gastrointestinal (GI) tract and urinary bladder. The α1-adrenergic receptors are member of the G_{q} protein-coupled receptor superfamily. Upon activation, a heterotrimeric G protein, G_{q}, activates phospholipase C (PLC). The mechanism of action involves interaction with calcium channels and changing the calcium content in a cell. For review, see Smith R. S. et al., Journal of Neurophysiology 102(2): 1103-14 (2009), which is incorporated by reference herein. Many cells possess these receptors.

α1-adrenergic receptors can be main receptor for fatty acids. For example, saw palmetto extract (SPE), widely used for the treatment of benign prostatic hyperplasia (BPH), has been reported to bind α1-adrenergic, muscarinic and 1,4-dihydropyridine (1,4-DHP) calcium channel antagonist receptors. See, Abe M., et al., Biol. Pharm. Bull. 32(4) 646-650 (2009), and Suzuki M. et al., Acta Pharmacologica Sinica 30:271-81 (2009), each of which is incorporated by reference herein. SPE includes a variety of fatty acids including lauric acid, oleic acid, myristic acid, palmitic acid and linoleic acid. Lauric acid and oleic acid can bind noncompetitively to cal-adrenergic, muscarinic and 1,4-DHP calcium channel antagonist receptors.

In certain embodiments, a permeation enhancer can be an adrenergic receptor blocker. The adrenergic receptor blocker can be a terpene (e.g. volatile unsaturated hydrocarbons found in the essential oils of plants, derived from units of isoprenes) or a C10-C22 alcohol or acid. In certain embodiments, the adrenergic receptor blocker can include farnesol, linoleic acid, arachidonic acid, docosahexanoic acid, eicosapentanoic acid, and/or docosapentanoic acid. The acid can be a carboxylic acid, phosphoric acid, sulfuric acid, hydroxamic acid, or derivatives thereof. The derivative can be an ester or amide. For example, the adrenergic receptor blocker can be a fatty acid or fatty alcohol.

The C10-C20 alcohol or acid can be an alcohol or acid having a straight C10-C22 hydrocarbon chain optionally containing at least one double bond, at least one triple bond, or at least one double bond and one triple bond; said hydrocarbon chain being optionally substituted with C₁₋₄ alkyl, C₂₋₄ alkenyl, C₂₋₄ alkynyl, C₁₋₄ alkoxy, hydroxyl, halo, amino, nitro, cyano, C₃₋₅ cycloalkyl, 3-5 membered heterocycloalkyl, monocyclic aryl, 5-6 membered heteroaryl, C₁₋₄ alkylcarbonyloxy, C₁₋₄ alkyloxycarbonyl, C₁₋₄ alkylcarbonyl, or formyl; and further being optionally interrupted by —O—, —N(R^{a})—, —N(R^{a})—C(O)—O—, —O—C(O)—N(R^{a})—, —N(R^{a})—C(O)—N(R^{b})—, or —O—C(O)—O—. Each of R^{a} and R^{b}, independently, is hydrogen, alkyl, alkenyl, alkynyl, alkoxy, hydroxylalkyl, hydroxyl, or haloalkyl.

The compositions described herein can include charged lipids or mixtures of charged lipids. As used herein, the term “charged lipid” is meant to include those lipids having one or two fatty acyl or fatty alkyl chains and a quaternary amino head group. The quaternary amine carries a permanent positive charge. The head group can optionally include an ionizable group, such as a primary, secondary, or tertiary amine that may be protonated at physiological pH. The presence of the quaternary amine can alter the pKa of the ionizable group relative to the pKa of the group in a structurally similar compound that lacks the quaternary amine (e.g., the quaternary amine is replaced by a tertiary amine) In some embodiments, a charged lipid is referred to as an “amino lipid.” By way of example, the compositions can include lipids that have quaternary amines and lipids that do have quaternary amines and lipids that do not have quaternary amines but do have a protonable amine group. A lipid including a quaternary amine can be in the form of a salt and can be prepared from a corresponding lipid that includes a tertiary amine. The tertiary amine can be converted to a quaternary amine by, e.g. alkylation with an appropriate alkyl halide. Other charged lipids can include those having alternative fatty acid groups and other quaternary groups, including those in which the alkyl substituents are different (e.g., N-ethyl-N-methylamino-, N-propyl-N-ethylamino- and the like). For those embodiments in which R1 and R2 are both long chain alkyl or acyl groups, they can be the same or different. In general, lipids (e.g., a charged lipid) having less saturated acyl chains are more easily sized, particularly when the complexes are sized below about 0.3 microns, for purposes of filter sterilization. Charged lipids containing unsaturated fatty acids with carbon chain lengths in the range of C10 to C20 are typical. Other scaffolds can also be used to separate the amino group (e.g., the amino group of the charged lipid) and the fatty acid or fatty alkyl portion of the charged lipid.

Fatty acids with a higher degree of unsaturation are effective candidates to enhance the permeation of drugs. Unsaturated fatty acids showed higher enhancement than saturated fatty acids, and the enhancement increased with the number of double bonds. A. Mittal, et al. Status of Fatty Acids as Skin Penetration Enhancers—A Review, Current Drug Delivery, 2009, 6, pp. 274-279. Position of double bond also affects the enhancing activity of fatty acids. Differences in the physicochemical properties of fatty acid which originate from differences in the double bond position most likely determine the efficacy of these compounds as skin penetration enhancers. Skin distribution increases as the position of the double bond is shifted towards the hydrophilic end. It has also been reported that fatty acid which has a double bond at an even number position more rapidly effects the perturbation of the structure of both the stratum corneum and the dermis than fatty acid which has double bond at an odd number position. Cis-unsaturation in the chain tends to increase activity. Id.

An adrenergic receptor interacter can be terpene. Hypotensive activity of terpenes in essential oils has been reported. See, Menezes I. A. et al., Z. Naturforsch. 65c:652-66 (2010), which is incorporated by reference herein. In certain embodiments, the permeation enhancer can be a sesquiterpene. Sesquiterpenes are a class of terpenes that consist of three isoprene units and have the empirical formula C₁₅H₂₄. Like monoterpenes, sesquiterpenes may be acyclic or contain rings, including many unique combinations. Biochemical modifications such as oxidation or rearrangement produce the related sesquiterpenoids.

An adrenergic receptor interacter can be an unsaturated fatty acid such as a linoleic acid. In certain embodiments, the permeation enhancer can be farnesol. Farnesol is a 15-carbon organic compound which is an acyclic sesquiterpene alcohol, which is a natural dephosphorylated form of farnesyl pyrophosphate. Under standard conditions, it is a colorless liquid. It is hydrophobic, and thus insoluble in water, but miscible with oils. Farnesol can be extracted from oils of plants such as citronella, neroli, cyclamen, and tuberose. It is an intermediate step in the biological synthesis of cholesterol from mevalonic acid in vertebrates. It has a delicate floral or weak citrus-lime odor and is used in performer. It has been reported that farnesol selectively kills acute myeloid leukemia blasts and leukemic cell lines in preference to primary hemopoietic cells. See, Rioja A. et al., FEBS Lett 467 (2-3): 291-5 (2000), which is incorporated by reference herein. Vasoactive properties of farnesyl analogues have been reported. See, Roullet, J.-B., et al., J. Clin. Invest., 1996, 97:2384-2390, which is incorporated by reference herein. Both Farnesol and N-acetyl-S-trans, trans-farnesyl-L-cysteine (AFC), a synthetic mimic of the carboxyl terminus of farnesylated proteins inhibited vasoconstriction in rat aortic rings.

In certain embodiments, an interacter can be an aporphine alkaloid. For example, an interacter can be a dicentrine.

In general, an interacter can also be a vasodilator or a therapeutic vasodilator. Vasodilators are drugs that open or widen blood vessels. They are typically used to treat hypertension, heart failure and angina, but can be used to treat other conditions as well, including glaucoma for example. Some vasodilators that act primarily on resistance vessels (arterial dilators) are used for hypertension, and heart failure, and angina; however, reflex cardiac stimulation makes some arterial dilators unsuitable for angina. Venous dilators are very effective for angina, and sometimes used for heart failure, but are not used as primary therapy for hypertension. Vasodilator drugs can be mixed (or balanced) vasodilators in that they dilate both arteries and veins and therefore can have wide application in hypertension, heart failure and angina. Some vasodilators, because of their mechanism of action, also have other important actions that can in some cases enhance their therapeutic utility or provide some additional therapeutic benefit. For example, some calcium channel blockers not only dilate blood vessels, but also depress cardiac mechanical and electrical function, which can enhance their antihypertensive actions and confer additional therapeutic benefit such as blocking arrhythmias.

Vasodilator drugs can be classified based on their site of action (arterial versus venous) or by mechanism of action. Some drugs primarily dilate resistance vessels (arterial dilators; e.g., hydralazine), while others primarily affect venous capacitance vessels (venous dilators; e.g., nitroglycerine). Many vasodilator drugs have mixed arterial and venous dilator properties (mixed dilators; e.g., alpha-adrenoceptor antagonists, angiotensin converting enzyme inhibitors), such as phentolamine.

It is more common, however, to classify vasodilator drugs based on their primary mechanism of action. The figure to the right depicts important mechanistic classes of vasodilator drugs. These classes of drugs, as well as other classes that produce vasodilation, include: alpha-adrenoceptor antagonists (alpha-blockers); Angiotensin converting enzyme (ACE) inhibitors; Angiotensin receptor blockers (ARBs); beta₂-adrenoceptor agonists (β₂-agonists); calcium-channel blockers (CCBs); centrally acting sympatholytics; direct acting vasodilators; endothelin receptor antagonists; ganglionic blockers; nitrodilators; phosphodiesterase inhibitors; potassium-channel openers; renin inhibitors.

In general, the active or inactive components or ingredients can be substances or compounds that create an increased blood flow or flushing of the tissue to enable a modification or difference (increase or decrease) in transmucosal uptake of the API(s), and/or have a positive or negative heat of solution which are used as aides to modify (increase or decrease) transmucosal uptake.

The pharmaceutical composition can be a spray, gum, gel, cream, tablet, liquid or film. The composition can include textures, for example, at the surface, such as microneedles or micro-protrusions. Recently, the use of micron-scale needles in increasing skin permeability has been shown to significantly increase transdermal delivery, including and especially for macromolecules. Most drug delivery studies have emphasized solid microneedles, which have been shown to increase skin permeability to a broad range of molecules and nanoparticles in vitro. In vivo studies have demonstrated delivery of oligonucleotides, reduction of blood glucose level by insulin, and induction of immune responses from protein and DNA vaccines. For such studies, needle arrays have been used to pierce holes into skin to increase transport by diffusion or iontophoresis or as drug carriers that release drug into the skin from a microneedle surface coating. Hollow microneedles have also been developed and shown to microinject insulin to diabetic rats. To address practical applications of microneedles, the ratio of microneedle fracture force to skin insertion force (i.e. margin of safety) was found to be optimal for needles with small tip radius and large wall thickness. Microneedles inserted into the skin of human subjects were reported as painless. Together, these results suggest that microneedles represent a promising technology to deliver therapeutic compounds into the skin for a range of possible applications. Using the tools of the microelectronics industry, microneedles have been fabricated with a range of sizes, shapes and materials. Microneedles can be, for example, polymeric, microscopic needles that deliver encapsulated drugs in a minimally invasive manner, but other suitable materials can be used.

Applicants have found that microneedles could be used to enhance the delivery of drugs through the oral mucosa, particularly with the claimed compositions. The microneedles create micron sized pores in the oral mucosa which can enhance the delivery of drugs across the mucosa. Solid, hollow or dissolving microneedles can be fabricated out of suitable materials including, but not limited to, metal, polymer, glass and ceramics. The microfabrication process can include photolithography, silicon etching, laser cutting, metal electroplating, metal electro polishing and molding. Microneedles could be solid which is used to pretreat the tissue and is removed before applying the film. The drug loaded polymer film described in this application can be used as the matrix material of the microneedles itself. These films can have microneedles or micro protrusions fabricated on its surface which will dissolve after forming microchannels in the mucosa through which drugs can permeate through.

The term “film” can include films and sheets, in any shape, including rectangular, square, or other desired shape. A film can be any desired thickness and size. In preferred embodiments, a film can have a thickness and size such that it can be administered to a user, for example, placed into the oral cavity of the user. A film can have a relatively thin thickness of from about 0.0025 mm to about 0.250 mm, or a film can have a somewhat thicker thickness of from about 0.250 mm to about 1.0 mm. For some films, the thickness may be even larger, i.e., greater than about 1.0 mm or thinner, i.e., less than about 0.0025 mm. A film can be a single layer or a film can be multi-layered, including laminated or multiple cast films.

Oral dissolving films can fall into three main classes: fast dissolving, moderate dissolving and slow dissolving. Fast dissolving films can dissolve in about 1 second to about 30 seconds in the mouth, or about 30 seconds to 1 minute in the mouth. Moderate dissolving films can dissolve in about 1 to about 30 minutes in the mouth, and slow dissolving films can dissolve in more than 30 minutes in the mouth. As a general trend, fast dissolving films can include (or consist of) low molecular weight hydrophilic polymers (e.g., polymers having a molecular weight between about 1,000 to 9,000, or polymers having a molecular weight up to 200,000). In contrast, slow dissolving films generally include high molecular weight polymers (e.g., having a molecular weight in millions). Moderate dissolving films can tend to fall in between the fast and slow dissolving films.

It can be preferable to use films that are moderate dissolving films. Moderate dissolving films can dissolve rather quickly, but also have a good level of mucoadhesion. Moderate dissolving films can also be flexible, quickly wettable, and are typically non-irritating to the user. Such moderate dissolving films can provide a quick enough dissolution rate, most desirably between about 1 minute and about 20 minutes, while providing an acceptable mucoadhesion level such that the film is not easily removable once it is placed in the oral cavity of the user. This can ensure delivery of a pharmaceutically active component to a user.

The pharmaceutical composition film can be manufactured with an occlusive layer and an active layer, with a suitable formulation. In one example, Applicants manufactured a film with an occlusive layer and an active layer. An occlusive layer can include, for example, an appropriate about of a cellulosic polymer, cellulose, a thickener, a polyol compound, a liquid vehicle (e.g., peceol), a taste additive or taste masking agent, and/or color additive(s). An active layer can include, e.g., an active pharmaceutical ingredient (in this case, octreotide), a water soluble component or resin (such as Sentry Polyox), a taste additive or taste masking agent, such as a sugar or sugar substitute, and a permeation enhancer (in this case, a surfactant).

### Pharmaceutically Active Component

A pharmaceutical composition can include one or more pharmaceutically active components. The pharmaceutically active component can be a single pharmaceutical component or a combination of pharmaceutical components. The pharmaceutically active component can be an anti-inflammatory analgesic agent, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, an antirheumatic, an antihypertensive, a bronchodilator, an anticholinergic, an anti-anxiety drug, an antiemetic compound, a hormone, a peptide, a protein or a vaccine. The pharmaceutically active component can be the compound, pharmaceutically acceptable salt of a drug, a prodrug, a derivative, a drug complex or analog of a drug. The term “prodrug” refers to a biologically inactive compound that can be metabolized in the body to produce a biologically active drug.

### Octreotide

In one example, the pharmaceutically active component can be a peptide, such as a cyclic peptide. A pharmaceutically active component can mimic a natural hormone. A pharmaceutically active component can be octreotide, which is an octapeptide that mimics natural somatostatin pharmacologically, but is a more potent an inhibitor of growth hormone glucagon and insulin than the natural hormone. Octreotide is used for the treatment of growth hormone producing tumors (acromegaly and gigantism), pituitary tumors that secrete thyroid stimulating hormone (thyrotropinoma), diarrhea and flushing episodes associated with carcinoid syndrome, and diarrhea in people with vasoactive intestinal peptide-secreting tumors (VIPomas). Octreotide is often given as an infusion for management of acute hemorrhage from esophageal varices in liver cirrhosis on the basis that it reduces portal venous pressure, though current evidence suggests that this effect is transient and does not improve survival. Octreotide is used in nuclear medicine imaging by labelling with indium-111 (Octreoscan) to noninvasively image neuroendocrine and other tumours expressing somatostatin receptors. More recently, it has been radiolabelled with carbon-11 as well as gallium-68, enabling imaging with positron emission tomography (PET), which provides higher resolution and sensitivity. Octreotide can also be labelled with a variety of radionuclides, such as yttrium-90 or lutetium-177, to enable peptide receptor radionuclide therapy (PRRT) for the treatment of unresectable neuroendocrine tumors. Octreotide can also be used in the treatment of Acromegaly, a disorder of excessive growth hormone (GH). Octreotide, being a somatostatin analog, inhibits the release of GH from the pituitary gland through a process normally involved in negative feedback.

### Franz Diffusion Cell

A Franz diffusion cell is an apparatus used for ex vivo tissue permeation assay used in the formulation development to identify the most active permeation enhancer. The Franz diffusion cell apparatus consists of two chambers separated by a membrane of, for example, animal or human skin. The test product is applied to the membrane via the top chamber. The bottom chamber contains fluid from which samples are taken at regular intervals for analysis to determine the amount of active that has permeated the membrane at set time points.

Referring to FIG. 1, a Franz diffusion cell 100 includes a donor compound 101, a donor chamber 102, a membrane 103, sampling port 104, receptor chamber 105, stir bar 106, and a heater/circulator 107.

Referring to FIG. 2, a pharmaceutical composition is a film 100 comprising a polymeric matrix 200, the pharmaceutically active component 300 being dispersed in the polymeric matrix. The film can include a permeation enhancer 400 which can be a surfactant, such as a cationic surfactant. The surfactant can also be a non-ionic or anionic surfactant, or a combination of cationic, non-ionic and/or anionic surfactants.

### Example 1—Performance Ranking of Octreotide Enhancers

Certain permeation enhancers typically cause precipitation of the pharmaceutically active component and/or the permeation enhancer. With respect to permeation enhancers, Applicants discovered that certain enhancers exhibited relatively improved compatibility with octreotide in terms of solubility without causing precipitation of octreotide and the permeation enhancer. These include certain cationic surfactants (e.g., DDTMAB, CTAB and BAC), certain anionic surfactants at higher concentrations (GDC, DOC), certain non-ionic surfactants (e.g., Polaxamer F127, Azone/DMCD, Labrasol, TDM), certain chelators (e.g., EDTA), certain cyclodextrins (e.g., dimethyl-cyclodextrin). The table below illustrates relative compatibility with octreotide and relative permeation ranking.

TABLE 1

| | | | Permeation Ranking |
|---|---|---|---|
| | | Octreotide | 0-1-2-3 |
| Enhancer type | Examples | Compatibility | None-Low-Med-High |
| Fatty acid | Oleic acid | No | 0 |
| Cyclodextrins | Dimethyl-cyclodextrin | Yes | 1 |
| | Beta-cyclodextrin | No | 0 |
| Chelators | EDTA | Yes | 1 |
| | EGTA | No | 0 |
| Surfactants: nonionic | Polaxamer F127 | Yes | 1 |
| | Azone/DMCD | Yes | 2 |
| | Peceol | No | 0 |
| | Labrasol | Yes | 0 |
| | TDM | Yes | 1 |
| Surfactants: anionic | GDC* | Yes | 2 |
| | DOC* | Yes | 1 |
| Surfactants: cationic | Dodecyltrimethylammonium | Yes | 3 |
| | bromide (DDTMAB) | | |
| | Hexadecyltrimethylammonium | Yes | 3 |
| | bromide (HDTMAB or | | |
| | CTAB) | | |
| | Benzalkonium chloride (BAC) | Yes | 3 |

*10% w/w Octreotide soluble after vigorous stirring

As shown in Table 1, cationic surfactants surprisingly exhibited both strong octreotide compatibility and permeation enhancement, with a rank score of 3 (high permeation). Thus, the use of one or a combination cationic surfactants, along with octreotide compatibility would deliver enhanced permeation of octreotide to a subject. As an alternative, any of the cationic surfactants can also be combined with any of the rank 2 or rank 1 enhancers (e.g., GDC, azone, EDTA, and dimethylcyclodextrin) that also exhibit octreotide compatibility. This would provide a pharmaceutical composition that provides enhanced delivery of octreotide to a subject.

### Example 2

Applicants also studied the following permeation enhancers and concentration of enhancers and compared average flux obtained from the ex vivo permeation model, as shown in Table 2 below.

TABLE 2

| Permeation enhancer | | | | |
|---|---|---|---|---|
| (frozen buccal tissue, dermatomed to | | Average flux | | PE |
| Membrane thickness of 300 um) | Wt % | (ug/cm2*min) | Std Dev | type |
| Glycine Betaine Ester C12 | 5 | 3.14 | 1.4 | CS |
| Dodecyltrimethylammonium bromide | 5 | 2.74 | 1.06 | CS |
| Decyltrimethylammonium bromide | 5 | 1.94 | 0.60 | CS |
| Benzalkonium chloride | 5 | 1.88 | 1.26 | CS |
| Dodecyltrimethylammonium bromide | 0.5 | 1.69 | 0.36 | CS |
| Dodecyltrimethylammonium bromide | 1 | 1.66 | 0.67 | CS |
| Hexadecyltrimethylammonium Bromide | 1 | 1.23 | 1.03 | CS |
| EDTA | 2 | 1.12 | 0.90 | C |
| Azone/Dimethylcyclodextrin | 5.5 | 1.08 | 0.80 | NS |
| Benzalkonium chloride | 1 | 1.03 | 0.40 | CS |
| Benzalkonium chloride | 0.1 | 0.89 | 0.90 | CS |
| Azone/Dmethylchyclodextrin | 2.5 | 0.87 | 0.72 | NS |
| GDC | 10 | 0.85 | 0.31 | AS |
| Lysalbinic acid/Dimethyl cyclodextrin | 5.5 | 0.77 | 1.25 | CS |
| Glycine Betaine Ester C16 | 5 | 0.69 | 0.13 | CS |
| Dodecyltrimethylammonium bromide + | 0.5 | 0.66 | 0.50 | CS |
| DMCD | | | | |
| L-α-Lysophosphatidyl choline | 1 | 0.59 | 0.32 | NS |
| Spermine HCl | 10 | 0.58 | 1.23 | MA |
| Dimethyl Cyclodextrin (DMCD) | 5 | 0.53 | 0.65 | IC |
| Glycine Betaine Ester C18 (Oleyl) | 5 | 0.51 | 0.66 | CS |
| DOC | 10 | 0.49 | 0.15 | AS |
| DOC | 10 | 0.41 | 0.19 | AS |
| Tetrahexylammonium bromide | 5 | 0.38 | 0.47 | CS |
| Dimethylcyclodextrin (clove pretreatment) | 5 | 0.36 | 0.16 | IC |
| EDTA | 2 | 0.36 | 0.31 | C |
| pluronic F127 | 2 | 0.29 | 0.16 | NS |
| benzalkonium chloride | 0.01 | 0.27 | 0.34 | CS |
| Labrafil M2125 CS | 5 | 0.27 | 0.41 | NS |
| Dodecyltrimethylammonium bromide + | 0.1/2 | 0.25 | 0.31 | CS |
| EDTA | | | | |
| Cholesterol amine | 10 | 0.25 | 0.40 | CS |
| pluronic F127 | 10 | 0.25 | 0.15 | NS |
| Decyltrimethylammonium bromide | 1 | 0.22 | 0.27 | CS |
| TDM | 2 | 0.21 | 0.25 | NS |
| Dodecyltrimethylammonium bromide | 0.1 | 0.20 | 0.32 | CS |
| Cetyl Pyridinium Chloride (CPC) | 5 | 0.20 | 0.13 | CS |
| Dimethylhexadecylamine pH 6.5 | 2 | 0.20 | 0.23 | CS |
| Dimethylhexadecylamine | 5 | 0.20 | 0.23 | CS |
| Linoleic acid/DMCD | 2.5 | 0.18 | 0.18 | FA |
| Brij 58 | 10 | 0.18 | 0.20 | NS |
| Dimethylhexadecylamine | 1 | 0.18 | 0.24 | CS |
| Hexyltrimethylammonium bromide | 1 | 0.17 | 0.28 | CS |
| Lysalbinic acid | 1 | 0.17 | 0.13 | S |
| No Permeation enhancer | 0 | 0.17 | 0.20 | |
| Hexyltrimethylammonium bromide | 5 | 0.16 | 0.14 | CS |
| Hydroxypropylcyclodextrin | 5 | 0.15 | 0.26 | IC |
| Cetyl betine | 5 | 0.15 | 0.09 | NS |
| PECEOL | 10 | 0.14 | 0.13 | NS |
| TDM | 5 | 0.14 | 0.17 | NS |
| L-α-Lysophosphatidyl choline | 2 | 0.14 | 0.15 | NS |
| Miltefosine (Hexadecyl Phosphatidyl | 7.5 | 0.14 | 0.17 | Z |
| Choline) | | | | |
| Phenyl piperazine | 5 | 0.14 | 0.15 | SM |
| GDC | 0.1 | 0.14 | 0.24 | AS |
| None (clove pretreatment) | 0 | 0.13 | 0.06 | |
| Brij 58 | 2 | 0.13 | 0.19 | NS |
| Labrasol | 5 | 0.12 | 0.19 | NS |
| Oleic acid/DMCD | 5.5 | 0.10 | 0.07 | FA |
| Maisine (Glycerol MonoLinoleate) | 10 | 0.10 | 0.12 | NS |
| Octyl pyrroldone | 5 | 0.10 | 0.10 | SM |
| Octyltrimethylammonium bromide | 1 | 0.10 | 0.19 | CS |
| EDTA | 10 | 0.10 | 0.13 | C |
| 1-Methyl 2-Pyrrolidone | 5 | 0.08 | 0.05 | SM |
| benzalkonium chloride | 0.05 | 0.08 | 0.05 | CS |
| Cetyl betaine | 10 | 0.07 | 0.05 | NS |
| poloxamer 124 Kollisolv P124 | 5 | 0.07 | 0.07 | SM |
| Dimethyl isosorbide | 5 | 0.07 | 0.04 | SM |
| sodium salicylate/DMCD | 2.2 | 0.06 | 0.07 | SM |
| Labrafil M 1944 CS | 5 | 0.04 | 0.05 | NS |
| Octyltrimethylammonium bromide | 5 | 0.03 | 0.03 | CS |
| clove | 0.5 | 0.03 | 0.03 | SM |
| M2125 Labrafil (Clove pretreatment) | 5 | 0.02 | 0.04 | NS |
| DOC/propylene glycol | 0.1/15 | 0.02 | 0.01 | AS |
| Lysalbinic acid | 5 | 0.01 | 0.01 | S |
| Clove | 1 | 0.01 | 0.02 | SM |
| β-Cyclodextrin | 1 | 0.01 | 0.01 | IC |
| oleic acid | 5 | 0.01 | 0.01 | FA |
| Cinnamaldehyde | 3 | 0.00 | 0.00 | SM |
| Cinnamaldehyde | 1 | 0.00 | 0.00 | SM |

Legend
AS Anionic surfactant
CS Cationic surfactant
Z Zwitterionic
SM Small molecule
NS Neutral Surfactant
FA Fatty Acid
C Chelator
MA MultiAmine
IC Inclusion Complex

As can be seen above, glycine betaine alkyl esters and dodecyltrimethylammonium bromide provided surprisingly enhanced average flux. GDC, decyltrimethyammonium bromide, azone and benzalkonium chloride (BAC) also delivered enhanced average flux results.
Exemplary structures of these enhancers are provided below

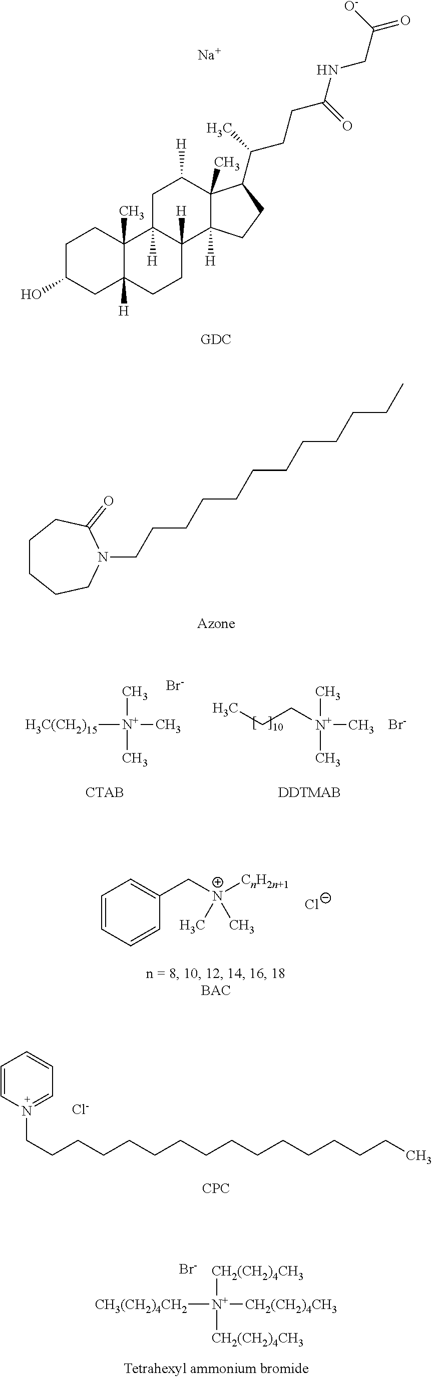

### Example 3

### DDTMAB Permeation Activity

Referring to FIG. 3, Applicants studied the effect of octreotide concentration on permeation with 5 wt % DDTMAB as an enhancer. The graph shows flux (μg/cm²*min) as a function of time. The square data points indicate 3 mg octreotide with DDTMAB enhancer. The diamond data points indicate 1.5 mg octreotide with DDTMAB enhancer. The cross-hatched data points indicate 0.6 mg octreotide with DDTMAB enhancer. The triangular data points refer to the 0.3 mg octreotide with DDTMAB enhancer.

As shown in the graph, the 5% DDTMAB approached up to 0.5 flux, including greater than 0.1, greater than 0.2, greater than 0.3, greater than 0.4, and about 0.5 flux in about 50-100 minutes, including greater than 50 minutes, greater than 60 minutes, greater than 70 minutes, greater than 80 minutes, greater than 90 minutes, and about 100 minutes. A flux of up about 2-2.5 in about 175 mins for 3 mg octreotide. A flux of up to 1.5 was obtained in about 175 mins for 1.5 mg octreotide. The lower concentrations required at about 125 minutes to approach a 0.25 flux. In sum, the data indicates that at constant DDTMAB concentration the permeation depends on octreotide concentration.

Referring to FIG. 4, the octreotide permeation follows Fick's first law of diffusion. Assuming the drug concentration in the donor compartment is constant and that in the receiver compartment, it is zero, the data points show a linear relationship between flux and drug concentration. Flux at approximately 0.500 ug/cm2*min was achieved at about octreotide concentration of 5 mg/ml. Flux at and above 2.0-2.5 ug/cm2*min, including greater than 2.0, greater than 2.1, greater than 2.2, greater than 2.3, greater than 2.4, and about 2.5 ug/cm2*min was reached between an octreotide concentration of 15-20 mg/ml including greater than 15 mg/ml, greater than 16 mg/ml, greater than 17 mg/ml, greater than 18 mg/ml, greater than 19 mg/ml and about 20 mg/ml.

### Example 4

Referring to FIG. 5, Applicants studied the structure-activity relationship of aliphatic trimethyl-ammonium bromide surfactants (e.g., n=5, 7, 9, 11, 15). The graph shows average flux as a function of time. The data shows that the permeation activity depends on the alkyl chain length with the optimum length being around 12. As the graph indicates, hexyl, octyl and decyl derivatives were not active at 1 wt %. Decyltrimethyl ammonium bromide (CMC˜1.7 wt %) was active at 5 wt %. At 1 wt %, C12 and C16 chain containing compounds are the most active.

The following data also indicates that a quaternary amine moiety is critical for activity.

TABLE 4

| Structure | Avg Flux (μg/cm²*min) |
|---|---|
| 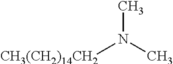 | 0.21 (5 wt %) |
| 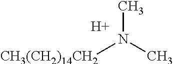 | 0.28 (2 wt %) |
| 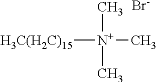 | 1.31 (1 wt %) |
| 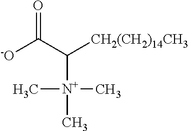 | 0.07 (10 wt %) |
| 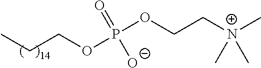 | 0.14 (7.5 wt %) |

### Example 5

Referring to FIG. 6, Applicants studied the microneedle impact on octreotide permeation with fresh porcine buccal tissue. In this study, Applicants used 0.75 mm microneedles and tissue approximately 1 μm in thickness, punched three times before being exposed to 3 mg octreotide. The graph indicates the average amount of octreotide permeated over time in minutes. The triangular data points show data with microneedles and 0% EDTA. The diamond data points show microneedles and 2% EDTA. The square data points were with no microneedles. The data show that with 2% EDTA, between 20-30 ug permeation, including greater than 20 ug, greater than 25 ug, and about 30 ug, less than 30 ug, less than 35 ug, and less than 20 ug, was achieved in approximately 1000-1250 minutes including greater than 1000 minutes, greater than 1100 minutes, greater than 1200 minutes, and about 1250 minutes.

Between 30-40 ug, including greater than 30 ug, greater than 35 ug, and about 40 ug, less than 40 ug, less than 35 ug, and less than 30 ug permeation was achieved in approximately 1250-1500 minutes including greater than 1250 minutes, greater than 1300 minutes, greater than 1350 minutes, greater than 1400 minutes, greater than 1450 minutes, and about 1500 minutes.

Applicants also discovered that microneedle application on buccal tissue does not cause octreotide degradation. As shown in the Table below, the buccal tissue was subjected to microneedle application (10 times) and incubated with octreotide solution (2 ml, 1 mg/ml) for 4 hours at 37 degrees C.

TABLE 5

| Sample | % Remaining |
|---|---|
| Octreotide control | 100 |
| Octreotide + Tissue | 86 |
| Octreotide + Tissue with microneedle | 98 |

### Example 6

Referring to FIG. 7, the graph shows the result of a POC study in mini pigs using a test solution of 12 mg octreotide in 500 μl PBS buffer and 5 wt % dodecyl trimethyl ammonium bromide, after an exposure time of 2 hours. Solutions were placed after scraping off mucin using a spatula and then treating the area with microneedles (750 um for buccal and 500 um for sublingual). Methocel (40% in water was used as a glue to attach the holder keeping the solution on to the tissue. The test material was colored so that any loss of drug during the experimental set up could be easily monitored.

The circular data points indicate buccal space. The square data points indicate sublingual space. Presences of octreotide in blood was observed for all the animals.

The following is a summary of the mean data values reflected in FIG. 7: The data shows that DDTMAB is an effective permeation enhancer for octreotide in preclinical models. It also shows that absorption is more efficient through sublingual mucosa compared to buccal.

| | Tmax | Cmax | AUC | Bio- |
|---|---|---|---|---|
| | (mins/hrs) | (ng/mL) | (ng*hr/mL) | availability |
| 100 μg octreotide acetate | 4 mins | 31.0 | 14.8 | NA |
| I.V. | | | | |
| 12 mg octreotide acetate | 3 hrs | 4.2 | 19.8 | 1.11% |
| solution applied to | | | | |
| disrupted buccal mucosa | | | | |
| 12 mg octreotide acetate | 3 hrs | 25.6 | 106.4 | 5.97% |
| applied to disrupted | | | | |
| sublingual mucosa | | | | |

### Example 8

The pharmaceutical composition film can be manufactured with an occlusive layer and an active layer, with a suitable formulation. In one example, the occlusive layer can include an appropriate about of a cellulose, such as metalose 90-SH 4000, a thickener such as a cellulose ether, for example, methocel E 15, a polyol compound such as glycerin, peceol, a color additive, and/or a taste additive (FD&C). Referring to FIG. 8, this shows an image of an exemplary pharmaceutical composition film. In certain embodiments, the film has an aspect ratio suitable to dispense an appropriate amount of pharmaceutical ingredient in a buccal and/or sublingual space. For example, the aspect ratio approximately 1:1-1:2, including greater than 1:1.9, greater than 1:1.8, greater than 1:1.7, greater than 1:1.6, greater than 1:1.5, greater than 1:1.4, greater than 1:1.3, greater than 1:1.2, greater than 1:1.1, about 1:1, less than 1:1.2, less than 1:1.3, less than 1:1.4, less than 1:1.5, less than 1:1.6, less than 1:1.7, less than 1:1.8, and less than 1:1.9. In one example a film was manufactured with a width 22 mm, a length of 25.6 mm and a backing layer with a width of 3 mm.

Occlusive Layer

| Material | Wt % dry |
|---|---|
| Metalose 90-SH 4000 | 46.72 |
| Methocel E15 | 46.73 |
| Glycerin | 6.000 |
| Peceol | 0.500 |
| FD&C # | 0.050 |

Active Layer

| Material | Wt % dry |
|---|---|
| Octreotide | 24.00 |
| Sentry Polyox WSR N10 | 7.59 |
| Sentry Polyox WSR N80 | 22.78 |
| Maltitol | 5.79 |
| DDTMAB | 39.84 |

### Example 9

Referring to FIG. 9A, this graph indicates the concentration dependent activity of DDTMAB using 3 mg octreotide in PBS pH 7.4. The triangle data points indicate amount permeated with 5% DDTMAB as a permeation enhancer. The diamond data points indicate amount permeated with 1% DDTMAB as a permeation enhancer.

With 5% DDTMAB, the octreotide permeated after 6 hours was greater than 500 ug, including greater than 510 ug, greater than 520 ug, and greater than 530 ug f 240 ug. The steady state flux was 3.24±1.24 ug/(cm²*min). Moreover, with 5% DDTMAB, the amount permeated was in the range of 100-200 ug between 100-150 minutes.

Referring to FIG. 9B, this graph indicates the average amount of octreotide permeated over time for 3 mg octreotide in a bilayer film. The triangle data points indicate the bilayer film with DDTMAB enhancer. The diamond data points show the data with no enhancer. The data shows that with the DDTMAB enhancer, the amount of octreotide permeated over time was greater than 170 ug, including greater than 150 ug, greater than 160 ug, and greater than 100 ug±122 ug. The steady state flux was 1.0±0.45 ug/(cm²*min). In contrast, without the enhancer, the amount permeated was significantly lower, and less than 25 ug, including less than 20 ug and less than 15 ug. The data shows that with the enhancer, the amount permeated started to increase significantly, including greater than 25 ug and up to 150-200 ug, including greater than 25 ug, greater than 50 ug, greater than 75 ug, greater than 100 ug, greater than 150 ug, and greater than 200 ug, about 200 ug, less than 200 ug, less than 150 ug, less than 100 ug, less than 75 ug, less than 50 ug, and less than 25 ug. The therapeutic window can range from 100-350 minutes including greater than 100 minutes, more than 110 minutes, more than 120 minutes, more than 130 minutes, more than 150 minutes, more than 200 minutes, more than 250 minutes, more than 300 minutes, about 350 minutes, less than 350 minutes, less than 300 minutes, less than 250 minutes, less than 200 minutes, less than 150 minutes, and less than 130 minutes, less than 120 minutes, and less than 110 minutes.

### Example 10

Referring to FIG. 10, this graph indicates results from an in vivo study using sublingual films with and without microneedles. The octreotide plasma concentration is shown in ng/ml following sublingual or subcutaneous administration to male miniature swine. The circular data points indicate 100 microgram octreotide solution with subcutaneous administration. The square data points indicate 15 mg octreotide administered in a pharmaceutical composition film. The triangular data points indicate 15 mg octreotide in microneedle administration. The data shows that with the pharmaceutical composition film, octreotide concentration (in ng/ml) achieved a range of between 10-55 ng/ml, including more than 10 ng/ml, more than 15 ng/ml, more than 20 ng/ml, more than 25 ng/ml, more than 30 ng/ml, more than 35 ng/ml, more than 40 ng/ml, more than 45 ng/ml, more than 50 ng/ml, approximately 55 ng/ml, less than 55 ng/ml, less than 50 ng/ml, less than 45 ng/ml, less than 40 ng/ml, less than 35 ng/ml, less than 30 ng/ml, less than 25 ng/ml, less than 20 ng/ml, less than 15 ng/ml, and less than 10 ng/ml. These concentrations were achieved in approximately 50-100 minutes, including more than 5 minutes, more than 10 minutes, more than 15 minutes, more than 20 minutes, more than 25 minutes, more than 30 minutes, more than 35 minutes, more than 40 minutes, more than 45 minutes, more than 50 minutes, more than 60 minutes, more than 70 minutes, more than 80 minutes, more than 90 minutes, and more than 100 minutes, less than 200 minutes, less than 150 minutes, less than 100 minutes, less than 90 minutes, less than 80 minutes, less than 70 minutes, less than 60 minutes, less than 50 minutes, less than 45 minutes, less than 40 minutes, less than 35 minutes, less than 30 minutes.

| | Cmax | AUC | |
|---|---|---|---|
| | (ng/mL) | (ng*hr/mL) | Bioavailability |
| Solution (subcutaneous) | 1.2 | 204.9 | |
| Octreotide film | 31.8 | 7423.8 | 6.48% |
| Octreotide film after microneedle | 28.2 | 5958.2 | 5.2% |
| application | | | |

The above data shows that significant absorption of octreotide from a solution and film was achieved in vivo. No effect of microneedle (500 um) pretreatment was seen in this particular study.

### Example 11

The applicants determined that cationic surfactants with biodegradable properties will be less irritating to the mucosa than the non degradable one. These lipids can be degraded through acid/base hydrolysis or enzymatic means. Several lipids were designed and prepared by putting degradable linkers between the cationic group and the long alkyl chain. The applicants reasoned that the degradants will be more biocompatible if they are naturally occurring. For example the degraded products from Glycine Betaine alkyl esters will be Glycine betaine and a long chain alcohol. Glycine Betaine is s a naturally occurring intracellular organic osmolyte and should be biocompatible and non toxic. The fatty alcohol which is benign and naturally occurring will be converted to a naturally occurring fatty acid by enzyme long-chain alcohol dehydrogenease.

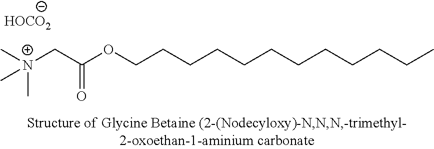 [SMILES: "HOCO2" ; CCCCCCCCCCCCOC(=O)C[N+](C)(C)C]

Applicants performed an ex vivo screening of permeation enhancers of octreotide delivery. Ex vivo porcine tissue was used, with a tissue thickness 300 um, 3 mg of octreotide and a time of up to 6 hours.

Referring to FIG. 11A, the results of a study of the concentration dependent permeation activity of glycine betaine ester-C12 are shown. The graph shows average flux (jg/cm²*min) as a function of time, with glycine betaine ester (GBE) as a permeation enhancer for octreotide.

The square data points show GBE 5 wt %. The triangular data points reflect GBE at 1 wt %. The cross-hatched data points reflect glycine betaine at 0.5 wt %. The permeation activity depends on the concentration of GBE C12.

As the graph shows, the average flux obtained was about 1 (μg/cm²*min) in less than 100 mins for GBE 5%, and in about 200 mins for GBE 1%, and about 270 mins for GBE 0.5%. The average flux of 1-3.5 (μg/cm²*min) was achieved between 50-400 minutes, including greater than 50 minutes, greater than 75 minutes, greater than 100 minutes, greater than 150 minutes, greater than 200 minutes, greater than 250 minutes, greater than 300 minutes, greater than 350 minutes, and about 400 minutes, less than 400 minutes, less than 350 minutes, less than 300 minutes, less than 250 minutes, less than 200 minutes, less than 150 minutes, less than 100 minutes, less than 75 minutes and less than 50 minutes.

Referring to FIG. 11B, the permeation activity is shown for glycine betaine esters with the effect of alkyl chains. The highest activity was observed for the GBE having C12 alkyl chain compared to one containing a C16 alkyl chain. The presence of unsaturation in the alkyl chain was found to have no effect on the activity. GBE-docecyl 5 wt % is indicated in square data points, as reaching a flux of 3-3.5 (μg/cm²*min) between 250-300 mins, including greater than 250, greater than 260, greater than 270, less than 280, and less than 290, and less than 300 minutes. GBE-hexadecyl 5 wt % was shown as reaching a flux of 3-3.5 between 250-300 mins, including greater than 250, greater than 260, greater than 270, less than 280, and less than 290, and less than 300 minutes. GBE-oleyl 5 wt % was shown as reaching a flux of 3-3.5 between 250-300 mins, including greater than 250, greater than 260, greater than 270, less than 280, and less than 290, and less than 300 minutes.

### Example 12

Referring to FIG. 12, the graph shows a comparison of the permeation activity of GBE C12 with DDTMAB (1 wt %). Both reach average flux (μg/cm2*min) of about 4 between 200-300 mins, including greater than 220 mins, greater than 230 mins, greater than 240 mins, greater than 250 minutes, greater than 260 mins, greater than 270 mins, less than 300 minutes, less than 290 mins, less than 280 mins, less than 270 mins and less than 260 mins. As indicated in the graph, the biodegradable permeation enhancer bears comparable efficiency with DDTMAB.

### Example 13

Referring to FIG. 13, the graph shows the results of cetyl pyridium chloride (CPC) as a permeation enhancer in average flux (μg/cm²*min) as a function of time (mins). CPC is a cationic surfactant where the quaternary nitrogen is part of a ring structure. As indicated, the diamond data points show CPC in 5 wt %. The square data points show CPC in 1 wt %. The triangular data points show CPC in 0.5 wt %. The cross-hatched data points show CPC in 0.1 wt %.

### Example 14

Referring to FIG. 14, the graph shows the results of tetrahexyl ammonium bromide which is a cationic surfactant with multiple long chain alkyl groups attached to the quaternary nitrogen as a permeation enhancer. The diamond data points show tetrahexyl ammonium bromide 5 wt %. The tetrahexyl ammonium bromide 5 wt % achieved flux of about 0.3-0.4 between 50-300 minutes. The square data points show tetrahexyl ammonium bromide 1 wt %. The tetrahexyl ammonium bromide 1 wt % achieve about 0.2 flux in between 250-300 minutes.

### Example 15

Referring to FIG. 15, the graph shows the results of benzalkonium chloride (BAC) as a permeation enhancer. The diamond data points show 5 wt % BAC. The square data points show 1 wt % BAC. The triangular data points show 0.1 wt % BAC. The lined-cross-hatched data points show 0.01% BAC. The cross-hatched data points show 0.05% wt BAC. The results show that the permeation activity is concentration dependent. An average flux of 1.8 pg/cm²*min was achieved with the use of 5 wt % BAC.

As the data indicates, the 5 wt % BAC achieved average flux of 1.5-2 in between 150-300 mins, including greater than 150, greater than 160, greater than 170, greater than 180, and greater than 200, greater than 220, greater than 240, greater than 250, greater than 260, greater than 260, greater than 270, greater than 280, less than 300, less than 290, less than 280, less than 270, less than 260 minutes.

The 1% BAC achieved a flux of about 1 in between 50-100 minutes, including greater than 50, greater than 60, greater than 70, greater than 80, greater than 90, less than 100, less than 90, less than 80, less than 70, and less than 60 minutes.

The 0.01% achieved a flux of about 1 in between 300-350 minutes, including greater than 300, greater than 310, greater than 320, greater than 330, greater than 340, greater than 350, less than 350, less than 340, less than 330, less than 320, and less than 310 minutes.

The 0.01% BAC achieved a flux of about 0.25 in between 300-350 minutes, including greater than 300, greater than 310, greater than 320, greater than 330, greater than 340, greater than 350, less than 350, less than 340, less than 330, less than 320, and less than 310 minutes.

The 0.05% BAC achieved a flux of about 0.25 in between 300-350 minutes, including greater than 300, greater than 310, greater than 320, greater than 330, greater than 340, greater than 350, less than 350, less than 340, less than 330, less than 320, and less than 310 minutes.

### Example 16

Referring to FIG. 16, the graph shows octreotide plasma concentration (ng/ml) vs. time profiles following sublingual or intravenous (IV) administration to male miniature swine.

| | | Octreotide |
|---|---|---|
| ID | Description | mg/strip |
| 3-1-1 | Original 40% w/w DDTMAB without | 11.5 |
| | Occlusive (single Layer) | |
| 8-1-1 | 40% w/w BAC with Occlusive (Bilayer) | 11.0 |
| 9-1-1 | Low 25% DDTMAB with Occlusive | 16.1 |
| | (Bilayer) | |

The circular data points indicate 100 microgram sandostatin (iv) (n=1). The square data points (8-1-1) indicate the 11 mg film (sublingual, average 32 mg benzalkonium chloride as permeation enhancer per film strip, n=4). This bilayer film has a slower dissolving backing layer in order to increase the residence time of drug in the sublingual space. The triangular data points (3-1-1) indicate a 11.5 mg single layer film (sublingual, average 35 mg Dodecyltrimethylammonium bromide as permeation enhancer per film strip, n=4). For 3-1-1, the active wet mass was coated on a thin film of placebo made with coat gap of 5 mil. The filled triangular data points (9-1-1) indicate a 16.1 mg bilayer film (sublingual, average 25 mg Dodecyltrimethylammonium bromide as permeation enhancer per film strip, n=4). The film size of 22×12.8 mm was used. The 16.1 mg film achieved octreotide concentration of about 10-30 mg/ml in about 50-150 minutes, including greater than 50, greater than 60, greater than 70, greater than 80, greater than 90, greater than 100, greater than 110, greater than 120, greater than 130, greater than 140, less than 150, less than 140, less than 130, less than 120, less than 110, less than 100, less than 90, less than 80, less than 70, less than 60 minutes.

The 11.5 mg film achieved octreotide concentration of about 10-18 in about 50-150 minutes, including greater than 50, greater than 60, greater than 70, greater than 80, greater than 90, greater than 100, greater than 110, greater than 120, greater than 130, greater than 140, less than 150, less than 140, less than 130, less than 120, less than 110, less than 100, less than 90, less than 80, less than 70, less than 60 minutes.

The 11 mg film achieved octreotide concentration of about 10-18 50-200 minutes, including greater than 50, greater than 60, greater than 70, greater than 80, greater than 90, greater than 100, greater than 110, greater than 120, greater than 130, greater than 140, less than 150, greater than 160, greater than 170, greater than 180, greater than 190, greater than 200, less than 190, less than 180, less than 170, less than 160, less than 150, less than 140, less than 130, less than 120, less than 110, less than 100, less than 90, less than 80, less than 70, less than 60 minutes.

Both benzalkonium chloride and dodecyltrimethylammonium bromide were found to be very efficient permeation enhancers with mean octreotide bioavailability of around 8-10%.

### Example 17

In one example, the inventors used High Intensity Focused Ultrasound (HIFU) as physical permeation enhancer. HIFU can have mechanical, cavitational or thermal effects on the tissue depending on the Ultrasound parameters such as intensity, duty cycle, pulse repetition frequency and the exposure time. In this experiment, the buccal tissue was subjected to HIFU for 30 seconds (power of 180 watts, duty cycle 5%, PRF of 10 Hz) octreotide permeation through the tissue was monitored for two hours in the ex vivo permeation model described earlier (except using full thickness tissue which includes connective tissue of the sub-mucosa). Almost 60 μg of octreotide was found to be permeated through the tissue whereas no octreotide permeation was observed without the HIFU application.

The target for matching 0.5 mg of the reference listed drug (RLD) in humans is 15 mg octreotide/40 mg BAC. The human study was designed with 2 arms: 10 mg/25 mg BAC and 15 mg/40 mg BAC. Referring to FIG. 17, the graph shows arm #1 of the human study (10 mg octreotide/25 mg BAC). The best profile with highest bioavailability was indicated with 3.1% bioavailability, the lowest 0.3% bioavailability, and the average/mean curve represented to be 1.3% bioavailability. The results unexpectedly show an order of magnitude difference for improved bioavailability. The two highest profiles show increased irritation, which is an indication of penetration, and validates that the enhancer is working. The four lowest profiles show only minor irritation (reddening). The transmucosal delivery of octreotide is evidenced by the fast onset of its availability in the plasma.

### Summary of Statistics of Transmucosal Absorption

Applicants have performed the first demonstrated delivery of peptides via transmucosal absorption. The relevant plasma concentrations showed a mean Cmax of 4600.55 pg/mL and a mean Tmax of 2.33 hr. This was a validation of the penetration enhancer mechanism. The level of irritation correlated with highest PK profiles. Referring to the table below, the summary of statistics and initial implications is presented.

| Descriptive | AUC₀₋ₜ | AUC_{0-inf} | Cₘₐₓ | Tₘₐₓ |
|---|---|---|---|---|
| Statistics | (hr*pg/mL) | (hr*pg/mL) | (pg/mL) | (hr) |
| N | 6 | 6 | 6 | 6 |
| Mean | 16784.08 | 17134.25 | 4600.55 | 2.33 |
| SD | 13709.27 | 13892.66 | 5153.84 | 1.37 |
| CV % | 81.68 | 81.08 | 112.03 | 58.6 |
| Min | 4609.42 | 4784.72 | 955.01 | 1.00 |
| Median | 12098.07 | 12469.44 | 2260.03 | 2.00 |
| Max | 42208.41 | 42925.03 | 14419.21 | 4.00 |

By comparison, Sandostatin (oral tablet) 0.1 mg SC injection reported Cmax=˜4100 pg/mL; AUC=13700 pg/mL and BA>1% (Max profile >3%) (data taken from Chiasma Overview September 2018). Surprisingly, the data shows that applicants' delivery via transmucosal absorption presents a Cmax that is comparable and AUC that is improved over the oral tablet.

### Degradation Studies

Referring to FIGS. 18A-18C, Applicants performed degradation studies on GBE-C₁₂ in biological media. The biologically relevant media tested were plasma (human BioIVT, K2-EDTA), esterase solution, simulated gastric fluid (pepsin/low pH), simulated intestinal fluid (pancreatin), and tissue homogenate (1 gram tissue+6 mL PBS solution→homogenizer (FastPrep)→centrifugation→supernatant). GBE-C₁₂ Ester was expected to be hydrolysed at 37 degrees C., to yielding glycine betaine+dodecanol under these conditions. As the graphs indicate in FIGS. 18A-18C, the studies showed that new permeation enhancer GBE-C₁₂ is highly biodegradable as quantified by the analysis of glycine betaine. It is not degradable in acidic environment as expected for an ester group. The results show that acid sensitive linkers such as acetals can be utilized.

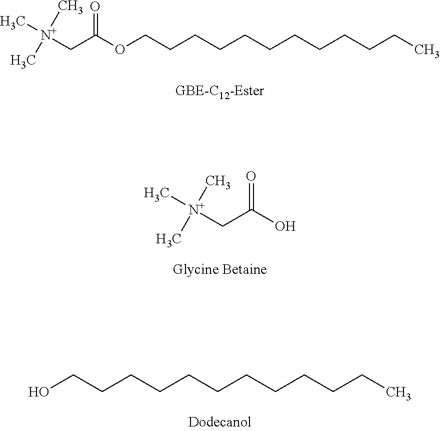

### Pharmaceutically Active Component

In some embodiments, more than one pharmaceutically active component may be included in the film. The pharmaceutically active components can be ace-inhibitors, anti-anginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, and combinations thereof.

### Pharmaceutical Film

A pharmaceutical composition film and/or its components for delivering octreotide can be water-soluble, water-swellable or water-insoluble. The term “water-soluble” can refer to substances that are at least partially dissolvable in an aqueous solvent, including but not limited to water. The term “water-soluble” may not necessarily mean that the substance is 100% dissolvable in the aqueous solvent. The term “water-insoluble” refers to substances that are not dissolvable in an aqueous solvent, including but not limited to water. A solvent can include water, or alternatively can include other solvents (preferably, polar solvents) by themselves or in combination with water.

The composition can include a polymeric matrix. Any desired polymeric matrix may be used, provided that it is orally dissolvable or erodible. Desirably, the dosage should have enough bioadhesion to not be easily removed and it should form a gel like structure when administered. They can be moderate-dissolving in the oral cavity and particularly suitable for delivery of pharmaceutically active components, although both fast release, delayed release, controlled release and sustained release compositions are also among the various embodiments contemplated.

The arrangement, order, or sequence of penetration enhancer(s) and active pharmaceutical ingredient(s)(API(s)) delivered to the desired mucosal surface can vary in order to deliver a desired pharmacokinetic profile. For example, one can apply the permeation enhancer(s) first by a film, by swab, spray, gel, rinse or by a first layer of a film then apply the API(s) by single film, by swab, or by a second layer of a film. The sequence can be reversed or modified, for example, by applying the API(s) first by film, by swab, or by a first layer of a film, and then applying the permeation enhancer(s) by a film, by swab, spray, gel, rinse or by a second layer of a film. In another embodiment, one may apply a permeation enhancer(s) by a film, and a drug by a different film. For example, the permeation enhancer(s) film positioned under a film containing the API(s), or the film containing the API(s) positioned under a film containing the permeation enhancer(s), depending on the desired pharmacokinetic profile.

For example, the penetration enhancer(s) can be used as a pretreatment alone or in combination with at least one API to precondition the mucosa for further absorption of the API(s). The treatment can be followed by another treatment with neat penetration enhancer(s) to follow the at least one API mucosal application. The pretreatment can be applied as a separate treatment (film, gel, solution, swab etc.) or as a layer within a multilayered film construction of one or more layers. Similarly, the pretreatment may be contained within a distinct domain of a single film, designed to dissolve and release to the mucosa prior to release of the secondary domains with or without penetration enhancer(s) or API(s). The active ingredient may then be delivered from a second treatment, alone or in combination with additional penetration enhancer(s). There may also be a tertiary treatment or domain that delivers additional penetration enhancer(s) and/or at least one API(s) or prodrug(s), either at a different ratio relative to each other or relative to the overall loading of the other treatments. This allows a custom pharmacokinetic profile to be obtained. In this way, the product may have single or multiple domains, with penetration enhancer(s) and API(s) that can vary in mucosal application order, composition, concentration, or overall loading that leads to the desired absorption amounts and/or rates that achieve the intended pharmacokinetic profile and/or pharmacodynamic effect.

The film format can be oriented such that no distinct sides, or such that the film has at least one side of a multiple layer film where the edges are co-terminus (having or meeting at a shared border or limit).

### Branched Polymers

The pharmaceutical composition film structured to deliver octreotide can include dendritic polymers comprise of highly branched macromolecules with various structural architectures and they include dendrimers, dendronised polymers (dendrigrafted polymers), Linear dendritic hybrids, multi-arm star polymers, and hyperbranched polymers.

Hyperbranched polymers are highly branched polymers but with imperfections in their structure. However they can be synthesized in a single step reaction which is an advantage over other dendritic structures and are therefore suitable for bulk volume applications. The properties of these polymers apart from their globular structure are the abundant functional groups, intramolecular cavities, low viscosity and high solubility. Dendritic polymers have been used in several drug delivery applications (Dendrimers as Drug Carriers: Applications in Different Routes of Drug Administration. J Pharm Sci, VOL. 97, 2008, 123-143.)

The dendritic polymers have internal cavities which can encapsulate drugs. The steric hindrance caused by the highly dense polymer chains might prevent the crystallization of the drugs. Therefore there may be an advantage of using branched polymers over linear ones in formulating physically metastable drugs prone to crystallization in a polymer matrix.

Examples of suitable dendritic polymers include poly(ether) based dendrons, dendrimers and hyperbranched polymers, poly(ester) based dendrons, dendrimers and hyperbranched polymers, poly(thioether) based dendrons, dendrimers and hyperbranched polymers, poly(amino acid) based dendrons dendrimers and hyperbranched polymers, poly(arylalkylene ether) based dendrons, dendrimers and hyperbranched polymers, poly(alkyleneimine) based dendrons, dendrimers and hyperbranched polymers, poly(amidoamine) based dendrons, dendrimers and hyperbranched polymers.

Other examples of hyperbranched polymers include poly(amines)s, polycarbonates, poly(ether ketone)s, polyurethanes, polycarbosilanes, polysiloxanes, poly(ester amine)s, poly(sulfone amine)s, poly(urea urethane)s and polyether polyols such as polyglycerols.

A pharmaceutical composition film can be produced by a combination of at least one polymer and a solvent, optionally including other components. The solvent may be water, a polar organic solvent including, but not limited to, ethanol, isopropanol, acetone, or any combination thereof. In some embodiments, the solvent may be a non-polar organic solvent, such as methylene chloride. The film may be prepared by utilizing a selected casting or deposition method and a controlled drying process. For example, the film may be prepared through controlled drying processes, which include application of heat and/or radiation energy to the wet film matrix to form a visco-elastic structure, thereby controlling the uniformity of content of the film. The controlled drying processes can include air alone, heat alone or heat and air together contacting the top of the film or bottom of the film or the substrate supporting the cast or deposited or extruded film or contacting more than one surface at the same time or at different times during the drying process (a bit clunky and may need to wordsmith here). Some of such processes are described in more detail in U.S. Pat. Nos. 8,765,167 and 8,652,378, which are incorporated by reference herein. Alternatively, the films may be extruded as described in U.S. Patent Publication No. 2005/0037055 A1, which is incorporated by reference herein.

A polymer included in the films may be water-soluble, water-swellable, water-insoluble, or a combination of one or more either water-soluble, water-swellable or water-insoluble polymers. The polymer may include cellulose, cellulose derivatives or gums. Specific examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water-insoluble polymers include, but are not limited to, ethyl cellulose, hydroxypropyl ethyl cellulose, cellulose acetate phthalate, hydroxypropyl methyl cellulose phthalate and combinations thereof. For higher dosages, it may be desirable to incorporate a polymer that provides a high level of viscosity as compared to lower dosages.

As used herein the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. In some embodiments, films formed from such water-soluble polymers may be sufficiently water-soluble to be dissolvable upon contact with bodily fluids.

Other polymers useful for incorporation into the films include biodegradable polymers, copolymers, block polymers and combinations thereof. It is understood that the term “biodegradable” is intended to include materials that chemically degrade, as opposed to materials that physically break apart (i.e., bioerodable materials). The polymers incorporated in the films can also include a combination of biodegradable or bioerodable materials. Among the known useful polymers or polymer classes which meet the above criteria are: poly(glycolic acid) (PGA), poly(lactic acid) (PLA), polydioxanes, polyoxalates, poly(alpha-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy)propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of .alpha.-amino acids, copolymers of .alpha.-amino acids and caproic acid, copolymers of .alpha.-benzyl glutamate and polyethylene glycol, copolymers of succinate and poly(glycols), polyphosphazene, polyhydroxy-alkanoates and mixtures thereof. The polymer matrix can include one, two, three, four or more components.

Although a variety of different polymers may be used, it is desired to select polymers that provide mucoadhesive properties to the film, as well as a desired dissolution and/or disintegration rate. In particular, the time period for which it is desired to maintain the film in contact with the mucosal tissue depends on the type of pharmaceutically active component contained in the composition. Some pharmaceutically active components may only require a few minutes for delivery through the mucosal tissue, whereas other pharmaceutically active components may require up to several hours or even longer. Accordingly, in some embodiments, one or more water-soluble polymers, as described above, may be used to form the film. In other embodiments, however, it may be desirable to use combinations of water-soluble polymers and polymers that are water-swellable, water-insoluble and/or biodegradable, as provided above. The inclusion of one or more polymers that are water-swellable, water-insoluble and/or biodegradable may provide films with slower dissolution or disintegration rates than films formed from water-soluble polymers alone. As such, the film may adhere to the mucosal tissue for longer periods or time, such as up to several hours, which may be desirable for delivery of certain pharmaceutically active components.

Desirably, an individual film dosage of the pharmaceutical film can have a small size, which is between about 0.0625-3 inch by about 0.0625-3 inch. The film size can also be greater than 0.25 inch, greater than 0.5 inch, greater than 1 inch, greater than 2 inches, about 3 inches, and greater than 3 inches, less than 3 inches, less than 2 inches, less than 1 inch, less than 0.5 inch, less than 0.0625 inch in at least one aspect, and greater than 0.0625 inch, greater than 0.5 inch, greater than 1 inch, greater than 2 inches, and greater than 3 inches, about 3 inches, less than 3 inches, less than 2 inches, less than 1 inch, less than 0.5 inch, less than 0.0625 inch in another aspect. The aspect ratio, including thickness, length, and width can be optimized by a person of ordinary skill in the art based on the chemical and physical properties of the polymeric matrix, the active pharmaceutical ingredient, dosage, enhancer, and other additives involved as well as the dimensions of the desired dispensing unit. The film dosage should have good adhesion when placed in the buccal cavity or in the sublingual region of the user. Further, the film dosage should disperse and dissolve at a moderate rate, most desirably dispersing within about 1 minute and dissolving within about 3 minutes. In some embodiments, the film dosage may be capable of dispersing and dissolving at a rate of between about 1 to about 30 minutes, for example, about 1 to about 20 minutes, or more than 1 minute, more than 5 minutes, more than 7 minutes, more than 10 minutes, more than 12 minutes, more than 15 minutes, more than 20 minutes, more than 30 minutes, about 30 minutes, and less than 30 minutes, less than 20 minutes, less than 15 minutes, less than 12 minutes, less than 10 minutes, less than 7 minutes, less than 5 minutes, and less than 1 minute. Sublingual rates may be shorter than buccal rates.

For instance, in some embodiments, the films may include polyethylene oxide alone or in combination with a second polymer component. The second polymer may be another water-soluble polymer, a water-swellable polymer, a water-insoluble polymer, a biodegradable polymer or any combination thereof. Suitable water-soluble polymers include, without limitation, any of those provided above. In some embodiments, the water-soluble polymer may include hydrophilic cellulosic polymers, such as hydroxypropyl cellulose and/or hydroxypropylmethyl cellulose. In some embodiments, one or more water-swellable, water-insoluble and/or biodegradable polymers also may be included in the polyethylene oxide-based film. Any of the water-swellable, water-insoluble or biodegradable polymers provided above may be employed. The second polymer component may be employed in amounts of about 0% to about 80% by weight in the polymer component, more specifically about 30% to about 70% by weight, and even more specifically about 40% to about 60% by weight.

Additives may be included in the films. Examples of classes of additives include preservatives, antimicrobials, excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers, anti-tacking agents, anti-static agents and mixtures thereof. These additives may be added with the pharmaceutically active component(s). The stabilizer can be a radical scavenger, an antioxidant, a buffering agent, an antimicrobial, an antifungal, a chelating agent or preservative, for example, sodium metabisulfite.

As used herein, the term “stabilizer” means an excipient capable of preventing aggregation or other physical degradation, as well as chemical degradation, of the active pharmaceutical ingredient, another excipient, or the combination thereof.

Stabilizers may also be classified as antioxidants, sequestrants, pH modifiers, emulsifiers and/or surfactants, and UV stabilizers. Antioxidants (i.e., pharmaceutically compatible compound(s) or composition(s) that decelerates, inhibits, interrupts and/or stops oxidation processes) include, in particular, the following substances: tocopherols and the esters thereof, sesamol of sesame oil, coniferyl benzoate of benzoin resin, nordihydroguaietic resin and nordihydroguaiaretic acid (NDGA), gallates (among others, methyl, ethyl, propyl, amyl, butyl, lauryl gallates), butylated hydroxyanisole (BHA/BHT, also butyl-p-cresol); ascorbic acid and salts and esters thereof (for example, acorbyl palmitate), erythorbinic acid (isoascorbinic acid) and salts and esters thereof, monothioglycerol, sodium formaldehyde sulfoxylate, sodium metabisulfite, sodium bisulfite, sodium sulfite, potassium metabisulfite, butylated hydroxyanisole, butylated hydroxytoluene (BHT), propionic acid. Typical antioxidants are tocopherol such as, for example, α-tocopherol and the esters thereof, butylated hydroxytoluene and butylated hydroxyanisole. The terms “tocopherol” also includes esters of tocopherol. A known tocopherol is α-tocopherol. The term “α-tocopherol” includes esters of α-tocopherol (for example, α-tocopherol acetate).

Sequestrants (i.e., any compounds which can engage in host-guest complex formation with another compound, such as the active ingredient or another excipient; also referred to as a sequestering agent) include calcium chloride, calcium disodium ethylene diamine tetra-acetate, glucono delta-lactone, sodium gluconate, potassium gluconate, sodium tripolyphosphate, sodium hexametaphosphate, and combinations thereof. Sequestrants also include cyclic oligosaccharides, such as cyclodextrins, cyclomannins (5 or more α-D-mannopyranose units linked at the 1,4 positions by α linkages), cyclogalactins (5 or more β-D-galactopyranose units linked at the 1,4 positions by β linkages), cycloaltrins (5 or more α-D-altropyranose units linked at the 1,4 positions by α linkages), and combinations thereof.
pH modifiers include acids (e.g., tartaric acid, citric acid, lactic acid, fumaric acid, phosphoric acid, ascorbic acid, acetic acid, succininc acid, adipic acid and maleic acid), acidic amino acids (e.g., glutamic acid, aspartic acid, etc.), inorganic salts (alkali metal salt, alkaline earth metal salt, ammonium salt, etc.) of such acidic substances, a salt of such acidic substance with an organic base (e.g., basic amino acid such as lysine, arginine and the like, meglumine and the like), and a solvate (e.g., hydrate) thereof. Other examples of pH modifiers include silicified microcrystalline cellulose, magnesium aluminometasilicate, calcium salts of phosphoric acid (e.g., calcium hydrogen phosphate anhydrous or hydrate, calcium, sodium or potassium carbonate or hydrogencarbonate and calcium lactate or mixtures thereof), sodium and/or calcium salts of carboxymethyl cellulose, cross-linked carboxymethylcellulose (e.g., croscarmellose sodium and/or calcium), polacrilin potassium, sodium and or/calcium alginate, docusate sodium, magnesium calcium, aluminium or zinc stearate, magnesium palmitate and magnesium oleate, sodium stearyl fumarate, and combinations thereof.

Examples of emulsifiers and/or surfactants include poloxamers or pluronics, polyethylene glycols, polyethylene glycol monostearate, polysorbates, sodium lauryl sulfate, polyethoxylated and hydrogenated castor oil, alkyl polyoside, a grafted water soluble protein on a hydrophobic backbone, lecithin, glyceryl monostearate, glyceryl monostearate/polyoxyethylene stearate, ketostearyl alcohol/sodium lauryl sulfate, carbomer, phospholipids, (C10-C20)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, and acylglutamates, quaternary ammonium salts (e.g., di-(C10-C24)-alkyl-dimethylammonium chloride or bromide), (C10-C24)-alkyl-dimethylethylammonium chloride or bromide, (C10-C24)-alkyl-trimethylammonium chloride or bromide (e.g., cetyltrimethylammonium chloride or bromide), (C10-C24)-alkyl-dimethylbenzylammonium chloride or bromide (e.g., (C12-C18)-alkyl-dimethylbenzylammonium chloride), N—(C10-C18)-alkyl-pyridinium chloride or bromide (e.g., N—(C12-C16)-alkyl-pyridinium chloride or bromide), N—(C10-C18)-alkyl-isoquinolinium chloride, bromide or monoalkyl sulfate, N—(C12-C18)-alkyl-polyoylaminoformylmethylpyridinium chloride, N—(C12-C18)-alkyl-N-methylmorpholinium chloride, bromide or monoalkyl sulfate, N—(C12-C18)-alkyl-N-ethylmorpholinium chloride, bromide or monoalkyl sulfate, (C16-C18)-alkyl-pentaoxethylammonium chloride, diisobutylphenoxyethoxyethyldimethylbenzylammonium chloride, salts of N,N-di-ethylaminoethylstearylamide and -oleylamide with hydrochloric acid, acetic acid, lactic acid, citric acid, phosphoric acid, N-acylaminoethyl-N,N-diethyl-N-methylammonium chloride, bromide or monoalkyl sulfate, and N-acylaminoethyl-N,N-diethyl-N-benzylammonium chloride, bromide or monoalkyl sulfate (in the foregoing, “acyl” standing for, e.g., stearyl or oleyl), and combinations thereof.

Examples of UV stabilizers include UV absorbers (e.g., benzophenones), UV quenchers (i.e., any compound that dissipates UV energy as heat, rather than allowing the energy to have a degradation effect), scavengers (i.e., any compound that eliminates free radicals resulting from exposure to UV radiation), and combinations thereof.

In other embodiments, stabilizers include ascorbyl palmitate, ascorbic acid, alpha tocopherol, butylated hydroxytoluene, buthylated hydroxyanisole, cysteine HCl, citric acid, ethylenediamine tetra acetic acid (EDTA), methionine, sodium citrate, sodium ascorbate, sodium thiosulfate, sodium metabi sulfite, sodium bisulfite, propyl gallate, glutathione, thioglycerol, singlet oxygen quenchers, hydroxyl radical scavengers, hydroperoxide removing agents, reducing agents, metal chelators, detergents, chaotropes, and combinations thereof. “Singlet oxygen quenchers” include, but are not limited to, alkyl imidazoles (e.g., histidine, L-camosine, histamine, imidazole 4-acetic acid), indoles (e.g., tryptophan and derivatives thereof, such as N-acetyl-S-methoxytryptamine, N-acetylserotonin, 6-methoxy-1,2,3,4-tetrahydro-beta-carboline), sulfur-containing amino acids (e.g., methionine, ethionine, djenkolic acid, lanthionine, N-formyl methionine, felinine, S-allyl cysteine, S-aminoethyl-L-cysteine), phenolic compounds (e.g., tyrosine and derivatives thereof), aromatic acids (e.g., ascorbate, salicylic acid, and derivatives thereof), azide (e.g., sodium azide), tocopherol and related vitamin E derivatives, and carotene and related vitamin A derivatives. “Hydroxyl radical scavengers” include, but are not limited to azide, dimethyl sulfoxide, histidine, mannitol, sucrose, glucose, salicylate, and L-cysteine. “Hydroperoxide removing agents” include, but are not limited to catalase, pyruvate, glutathione, and glutathione peroxidases. “Reducing agents” include, but are not limited to, cysteine and mercaptoethylene. “Metal chelators” include, but are not limited to, EDTA, EGTA, o-phenanthroline, and citrate. “Detergents” include, but are not limited to, SDS and sodium lauroyl sarcosyl. “Chaotropes” include, but are not limited to guandinium hydrochloride, isothiocyanate, urea, and formamide.

Useful additives can include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers.

The additional components can range up to about 80%, desirably about 0.005% to 50% and more desirably within the range of 1% to 20% based on the weight of all composition components including greater than 1%, greater than 5%, greater than 10%, greater than 20%, greater than 30%, greater than 40%, greater than 50%, greater than 60%, greater than 70%, about 80%, greater than 80%, less than 80%, less than 70%, less than 60%, less than 50%, less than 40%, less than 30%, less than 20%, less than 10%, less than 5%, about 3%, and less than 1%. Other additives can include anti-tacking, flow agents and opacifiers, such as the oxides of magnesium aluminum, silicon, titanium, etc. desirably in a concentration range of about 0.005% to about 5% by weight and desirably about 0.02% to about 2% based on the weight of all film components, including greater than 0.02%, greater than 0.2%, greater than 0.5%, greater than 1%, greater than 1.5%, greater than 2%, greater than 4%, about 5%, greater than 5%, less than 4%, less than 2%, less than 1%, less than 0.5%, less than 0.2%, and less then 0.02%. Other additives can include anti-tacking, flow agents and opacifiers, such as the oxides of magnesium aluminum, silicon, titanium, etc. desirably in a concentration range of about 0.01% to about 5% by weight and desirably about 0.02% to about 1% based on the weight of all film components.

In certain embodiments, the composition can include plasticizers, which can include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sugar alcohols, sorbitol, sodium diethylsulfosuccinate, triethyl citrate, tributyl citrate, phytoextracts, fatty acid esters, fatty acids, oils and the like, added in concentrations ranging from about 0.1% to about 40%, and desirably ranging from about 0.5% to about 20% based on the weight of the composition. There may further be added compounds to improve the texture properties of the film material such as animal or vegetable fats, desirably in their hydrogenated form. The composition can also include compounds to improve the textural properties of the product. Other ingredients can include binders which contribute to the ease of formation and general quality of the films. Non-limiting examples of binders include starches, natural gums, pregelatinized starches, gelatin, polyvinylpyrrolidone, methylcellulose, sodium carboxymethylcellulose, ethylcellulose, polyacrylamides, polyvinyloxoazolidone, and polyvinylalcohols.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable pharmaceutically active components may fit within the hydrophobic cavity, thereby producing an inclusion complex, which is soluble in water. Accordingly, the formation of the inclusion complex permits very insoluble and/or unstable pharmaceutically active components to be dissolved in water. A particularly desirable example of such agents are cyclodextrins, which are cyclic carbohydrates derived from starch. Other similar substances, however, are considered well within the scope of the present invention.

Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminium hydroxide. Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof, saccharin and its various salts such as the sodium salt; dipeptide based sweeteners such as aspartame, neotame, advantame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

Anti-foaming and/or de-foaming components may also be used with the films. These components aid in the removal of air, such as entrapped air, from the film-forming compositions. Such entrapped air may lead to non-uniform films. Simethicone is one particularly useful anti-foaming and/or de-foaming agent. The present invention, however, is not so limited and other anti-foam and/or de-foaming agents may suitable be used. Simethicone and related agents may be employed for densification purposes. More specifically, such agents may facilitate the removal of voids, air, moisture, and similar undesired components, thereby providing denser and thus more uniform films. Agents or components which perform this function can be referred to as densification or densifying agents. As described above, entrapped air or undesired components may lead to non-uniform films.

Any other optional components described in commonly assigned U.S. Pat. Nos. 7,425,292 and 8,765,167, referred to above, also may be included in the films described herein.

The film compositions further desirably contains a buffer so as to control the pH of the film composition. Any desired level of buffer may be incorporated into the film composition so as to provide the desired pH level encountered as the pharmaceutically active component is released from the composition. The buffer is preferably provided in an amount sufficient to control the release from the film and/or the absorption into the body of the pharmaceutically active component. In some embodiments, the buffer may include sodium citrate, citric acid, bitartrate and combinations thereof.

The pharmaceutical films described herein may be formed via any desired process. Suitable processes are set forth in U.S. Pat. Nos. 8,652,378, 7,425,292 and 7,357,891, the entire contents of which are incorporated by reference herein. In one embodiment, the film dosage composition is formed by first preparing a wet composition, the wet composition including a polymeric carrier matrix and a therapeutically effective amount of a pharmaceutically active component. The wet composition is cast into a film and then sufficiently dried to form a self-supporting film composition. The wet composition may be cast into individual dosages, or it may be cast into a sheet, where the sheet is then cut into individual dosages.

The pharmaceutical composition can adhere to a mucosal surface. The present invention finds particular use in the localized treatment of body tissues, diseases, or wounds which may have moist surfaces and which are susceptible to bodily fluids, such as the mouth, the vagina, organs, or other types of mucosal surfaces. The device carries a pharmaceutical, and upon application and adherence to the mucosal surface, offers a layer of protection and delivers the pharmaceutical to the treatment site, the surrounding tissues, and other bodily fluids. The device provides an appropriate residence time for effective drug delivery at the treatment site, given the control of erosion in aqueous solution or bodily fluids such as saliva, and the slow, natural erosion of the film concomitant or subsequent to the delivery.

The residence time of the device of the composition depends on the erosion rate of the water erodable polymers used in the formulation and their respective concentrations. The erosion rate may be adjusted, for example, by mixing together components with different solubility characteristics or chemically different polymers, such as hydroxyethyl cellulose and hydroxypropyl cellulose; by using different molecular weight grades of the same polymer, such as mixing low and medium molecular weight hydroxyethyl cellulose; by using excipients or plasticizers of various lipophilic values or water solubility characteristics (including essentially insoluble components); by using water soluble organic and inorganic salts; by using crosslinking agents such as glyoxal with polymers such as hydroxyethyl cellulose for partial crosslinking; or by post-treatment irradiation or curing, which may alter the physical state of the film, including its crystallinity or phase transition, once obtained. These strategies might be employed alone or in combination in order to modify the erosion kinetics of the device. Upon application, the pharmaceutical delivery device adheres to the mucosal surface and is held in place. Water absorption softens the device, thereby diminishing the foreign body sensation. As the device rests on the mucosal surface, delivery of the drug occurs. Residence times may be adjusted over a wide range depending upon the desired timing of the delivery of the chosen pharmaceutical and the desired lifespan of the carrier. Generally, however, the residence time is modulated between about a few seconds to about a few days. Preferably, the residence time for most pharmaceuticals is adjusted from about 5 seconds to about 24 hours. More preferably, the residence time is adjusted from about 5 seconds to about 30 minutes. In addition to providing drug delivery, once the device adheres to the mucosal surface, it also provides protection to the treatment site, acting as an erodable bandage. Lipophilic agents can be designed to slow down erodability to decrease disintegration and dissolution.

It is also possible to adjust the kinetics of erodability of the devices by adding excipients which are sensitive to enzymes such as amylase, very soluble in water such as water soluble organic and inorganic salts. Suitable excipients may include the sodium and potassium salts of chloride, carbonate, bicarbonate, citrate, trifluoroacetate, benzoate, phosphate, fluoride, sulfate, or tartrate. The amount added can vary depending upon how much the erosion kinetics is to be altered as well as the amount and nature of the other components in the device.

Emulsifiers typically used in the water-based emulsions described above are, preferably, either obtained in situ if selected from the linoleic, palmitic, myristoleic, lauric, stearic, cetoleic or oleic acids and sodium or potassium hydroxide, or selected from the laurate, palmitate, stearate, or oleate esters of sorbitol and sorbitol anhydrides, polyoxyethylene derivatives including monooleate, monostearate, monopalmitate, monolaurate, fatty alcohols, alkyl phenols, allyl ethers, alkyl aryl ethers, sorbitan monostearate, sorbitan monooleate and sorbitan monopalmitate.

The amount of pharmaceutically active component to be used depends on the desired treatment strength and the composition of the layers, although preferably, the pharmaceutical component comprises from about 0.001% to about 99%, more preferably from about 0.003 to about 75%, and most preferably from about 0.005% to about 50% by weight of the composition, including, more than 0.005%, more than 0.05%, more than 0.5%, more than 1%, more than 5%, more than 10%, more than 15%, more than 20%, more than 30%, about 50%, more than 50%, less than 50%, less than 30%, less than 20%, less than 15%, less than 10%, less than 5%, less than 1%, less than 0.5%, less than 0.05%, and less than 0.005%. The amounts of other components may vary depending on the drug or other components but typically these components comprise no more than 50%, preferably no more than 30%, most preferably no more than 15% by total weight of the device.

The thickness of the film may vary, depending on the thickness of each of the layers and the number of layers. As stated above, both the thickness and amount of layers may be adjusted in order to vary the erosion kinetics. Preferably, if the device has only two layers, the thickness ranges from 0.005 mm to 2 mm, preferably from 0.01 to 1 mm, and more preferably from 0.1 to 0.5 mm, including greater than 0.1 mm, greater than 0.2 mm, about 0.5 mm, greater than 0.5 mm, less than 0.5 mm, less than 0.2 mm, and less than 0.1 mm. The thickness of each layer may vary from 10 to 90% of the overall thickness of the layered device, and preferably varies from 30 to 60%, including greater than 10%, greater than 20%, greater than 30%, greater than 40%, greater than 50%, greater than 70%, greater than 90%, about 90%, less than 90%, less than 70%, less than 50%, less than 40%, less than 30%, less than 20%, and less than 10%. Thus, the preferred thickness of each layer may vary from 0.01 mm to 0.9 mm, and from 0.03 to 0.5 mm.

As one skilled in the art will appreciate, when systemic delivery. e.g., transmucosal or transdermal delivery is desired, the treatment site may include any area in which the film is capable of delivery and/or maintaining a desired level of pharmaceutical in the blood, lymph, or other bodily fluid. Typically, such treatment sites include the oral, esophageal, aural, ocular, anal, nasal, and vaginal mucosal tissue, as well as, the skin. If the skin is to be employed as the treatment site, then usually larger areas of the skin wherein movement will not disrupt the adhesion of the device, such as the upper arm or thigh, are preferred.

The pharmaceutical composition can also be used as a wound dressing. By offering a physical, compatible, oxygen and moisture permeable, flexible barrier which can be washed away, the film can not only protect a wound but also deliver a pharmaceutical in order to promote healing, asepty, scarification, to ease the pain or to improve globally the condition of the sufferer. Some of the: examples given below are well suited for an application to the skin or a wound. As one skilled in the art will appreciate, the formulation might require incorporating a specific hydrophilic/hygroscopic excipient which would help in maintaining good adhesion on dry skin over an extended period of time. Another advantage of the present invention when utilized in this manner is that if one does not wish that the film be noticeable on the skin, then no dyes or colored substances need be used. If, on the other hand, one desires that the film be noticeable, a dye or colored substance may be employed.

While the pharmaceutical composition can adhere to mucosal tissues, which are wet tissues by nature, it can also be used on other surfaces such as skin or wounds. The pharmaceutical film can adhere to the skin if prior to application the skin is wet with an aqueous-based fluid such as water, saliva, wound drainage or perspiration. The film can adhere to the skin until it erodes due to contact with water by, for example, rinsing, showering, bathing or washing. The film may also be readily removed by peeling without significant damage to tissue.

All references cited herein are hereby incorporated by reference herein in their entirety. Other embodiments are within the scope of the following claims.

What is claimed is:

1. A pharmaceutical composition, comprising:
a polymeric matrix;
a pharmaceutically active component including octreotide in the polymeric matrix; and
a permeation enhancer including a surfactant.

2. The pharmaceutical composition according to claim 1, wherein the surfactant is a cationic surfactant.

3. The pharmaceutical composition according to claim 1, wherein the surfactant includes a dodecyltrimethylammonium bromide.

4. The pharmaceutical composition according to claim 1, wherein the surfactant includes a glycine betaine ester.

5. The pharmaceutical composition according to claim 1, wherein the surfactant includes CTAB.

6. The pharmaceutical composition according to claim 1, wherein the surfactant includes BAC.

7. The pharmaceutical composition according to claim 1, wherein the surfactant includes CPC.

8. The pharmaceutical composition according to claim 1, wherein the surfactant is combined with a non-ionic or anionic surfactant.

9. The pharmaceutical composition according to claim 1, wherein the surfactant is combined with a chelator.

10. The pharmaceutical composition according to claim 1, wherein the surfactant is combined with a cyclodextrin.

11. The pharmaceutical composition according to claim 1, wherein the surfactant is combined with a fatty acid.

12. The pharmaceutical composition according to claim 1, wherein the permeation enhancer is biodegradable.

13. The pharmaceutical composition according to claim 1 having a suitable nontoxic, nonionic alkyl glycoside having a hydrophobic alkyl group joined by a linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced mucosal delivery, wherein the formulation of the compound with the transmucosal delivery-enhancing agents provides for increased bioavailability of the compound in a blood plasma of a subject.

14. The pharmaceutical composition according to claim 1, wherein the octreotide is delivered from a pharmaceutical film having an occlusive layer and an active layer.

15. The pharmaceutical composition according to claim 1, wherein the octreotide and permeation enhancer are embedded in an active layer of a pharmaceutical composition film.

16. The pharmaceutical composition according to claim 1 wherein the permeation activity of DDTMAB is concentration dependent.

17. The pharmaceutical composition according to claim 1, wherein the permeation enhancer is 5% wt DDTMAB.

18. The pharmaceutical composition according to claim 1, wherein the permeation enhancer is 1% wt DDTMAB.

19. The pharmaceutical composition according to claim 1, wherein the permeation enhancer is 0.5% wt DDTMAB.

20. The pharmaceutical composition according to claim 1, wherein the permeation enhancer is 0.1% wt DDTMAB.

21. The pharmaceutical composition according to claim 1, with a critical micelle concentration of 0.3%.

22. The pharmaceutical composition according to claim 1, wherein the permeation enhancer is 10% wt glycine betaine ester.

23. The pharmaceutical composition according to claim 1, wherein the permeation enhancer is 5% wt glycine betaine ester.

24. The pharmaceutical composition according to claim 1, wherein the permeation enhancer is 0.5% wt glycine betaine ester.

25. The pharmaceutical composition according to claim 1, wherein the permeation enhancer is 0.1% wt glycine betaine ester.

26. The pharmaceutical composition according to claim 1, having a therapeutic window 300 minutes or less.

27. The pharmaceutical composition according to claim 1, having a therapeutic window of 200 minutes or less.

28. The pharmaceutical composition according to claim 1, having a therapeutic window of 150 minutes or less.

29. The pharmaceutical composition according to claim 1, having a therapeutic window of 100 minutes or less.

30. The pharmaceutical composition according to claim 1, having a therapeutic window of 50 minutes or less.

31. The pharmaceutical composition according to claim 1, having a therapeutic window of 50-400 minutes.

32. The pharmaceutical composition according to claim 1, having 50-600 ug of octreotide permeation in a therapeutic window.

33. The pharmaceutical composition according to claim 1, wherein the polymeric matrix comprises at least one polymer selected from the group of: pullulan, polyvinyl pyrrolidone, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, ethylene oxide-propylene oxide co-polymers, collagen, albumin, poly-amino acids, polyphosphazenes, polysaccharides, chitin, chitosan, and derivatives thereof.

34. The pharmaceutical composition according to claim 1, further comprising a stabilizer.

35. The pharmaceutical composition according to claim 1, wherein the polymeric matrix comprises a dendritic polymer.

36. The pharmaceutical composition according to claim 1, wherein the polymeric matrix comprises a hyperbranched polymer.

37. A method of making a pharmaceutical composition of claim 1, comprising:
mixing a permeation enhancer including a surfactant with a pharmaceutically active component including octreotide and
embedding the pharmaceutically active component including octreotide in a pharmaceutical film.

38. A device comprising
a housing that holds an amount of a pharmaceutical composition, comprising:
a polymeric matrix;
a pharmaceutically active component including octreotide in the polymeric matrix; and
a permeation enhancer including a surfactant; and
an opening that dispenses a predetermined amount of the pharmaceutical composition.

39. The pharmaceutical composition of claim 1, wherein the surfactant has the following structure: 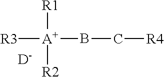
wherein:
A is either nitrogen or phosphorus;
C is a cleavable linkage;
B is a group connecting A with C and is an alkylene, alkenylene, cycloalkylene or aralkylene group or a derivatives thereof optionally containing one or more hetero atoms;
each of R¹, R² and R³, independently, is selected from the group consisting of hydrogen, alkyl, alkenyl, alkynyl, cycloalkyl and aralkyl group optionally having one or more heteroatoms;
R⁴ is selected from the group consisting of alkyl, alkenyl, alkynyl, cycloalkyl and aralkyl group optionally having one or more heteroatoms;
D− is an anionic counter ion to A′.

40. The pharmaceutical composition of claim 39, wherein each of R¹, R² and R³, independently, is a C₁₋₁₀ alkyl, C₂₋₁₀ alkenyl, C₂₋₁₀ alkynyl, C₃₋₁₀ cycloalkyl, C₄₋₁₀ aralkyl group or derivative thereof optionally having one or more heteroatoms.

41. The pharmaceutical composition of claim 39 wherein B is a C₁₋₂₀ alkylene, C₂₋₂₀ alkenylene, C₂₋₂₀ alkynylene, C₃₋₂₀ cycloalkylene, C₄₋₂₀ aralkylene group or derivative thereof optionally having one or more heteroatoms.

42. The pharmaceutical composition of claim 39 wherein R⁴ is a C₁₋₃₀ alkyl, C₂₋₃₀ alkenyl, C₂₋₃₀ alkynyl, C₃₋₃₀ cycloalkyl, C₄₋₃₀ aralkyl group or derivative thereof optionally having one or more heteroatoms.

43. The pharmaceutical composition of claim 39 wherein C is a degradable group through acid/base hydrolysis, enzymatic reaction or radical cleavage.

44. The pharmaceutical composition of claim 39 wherein C is selected from the group consisting of a carbonate linkage, an amide linkage, an ester linkage, acetal linkage, hemiacetal linkage, orthoester linkage, carbamide, sulphonate, phosphonate, thioester, urea, isocyanate linkages, hydrozone, disulfide linkages or any combination thereof.

45. The pharmaceutical composition of claim 39 wherein D is chloride, bromide, iodide, sulfate, sulfonate, carbonate, or hydroxide ion.

46. A method of treating a medical condition comprising:
administering a pharmaceutical composition including:
a polymeric matrix;
an effective amount of a pharmaceutically active component including octreotide in the polymeric matrix; and
a permeation enhancer including a surfactant.

47. The method of claim 46, wherein treating a medical condition includes inhibiting the release of growth hormone.

48. The method of claim 46, wherein the medical condition includes growth hormone producing tumors and pituitary tumors, diarrhea and flushing episodes associated with carcinoid syndrome, diarrhea associated with vasoactive intestinal peptide-secreting tumors, or acute hemorrhage from esophageal varices in liver cirrhosis.
